(19) United States
(12) Patent Application Publication
(10) Pub. No.: US 2011/0269716 A1
(43) Pub. Date: Nov. 3, 2011

(54) ANTIOXIDANT COMPOUNDS AND METHODS OF THEIR USE

(75) Inventors: Steven Bottle, Queensland (AU); Kathryn Fairfull-Smith, Queensland (AU); Farina Schill, Goettingen (DE)

(73) Assignee: Queensland University of Technology, Brisbane (AU)

(21) Appl. No.: 13/121,666

(22) PCT Filed: Sep. 30, 2009
(86) PCT No.: PCT/AU2009/001303

§ 371 Date: Jun. 13, 2011

(30) Foreign Application Priority Data
Sep. 30, 2008 (AU) 200890505077

(51) Int. Cl. A61K 31/675; A61P 39/06; C07D 209/44; C07F 9/572; C12N 5/071; A61K 31/4035

(52) U.S. Cl. 514/80; 435/375; 514/416; 548/482; 548/414

(57) ABSTRACT

The invention relates to antioxidant substituted isoindoline nitroxide compounds and their use in methods of treating or preventing diseases or disorders related to oxidative stress, methods of reducing oxidative stress and methods of protecting a subject from oxidative stress upon exposure to ionising radiation. Pharmaceutical compositions comprising the antioxidant compounds are also described.

### FIELD OF THE INVENTION

The present invention relates to antioxidant compounds and their use in methods of treating or preventing disorders or diseases related to oxidative stress. In particular, the antioxidant compounds are substituted isoindoline nitroxide compounds. Pharmaceutical compositions containing the antioxidant compounds are also described.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Nitroxides are stable free-radical species currently utilised in a variety of applications including use as antioxidants. Their redox and radical trapping properties can reduce levels of oxidative stress in cellular systems caused by reactive oxygen species (ROS). Nitroxides can also provide radio-protection towards ionising radiation.

The isoindoline nitroxide, 5-carboxy-1,1,3,3-tetramethyl-isoindolin-2-yloxyl (CTMIO) is known to have a protective effect on radiation-induced oxidative stress in cells affected with Ataxia Telangiectasia (A-T), a genetic disease characterised by neurodegeneration, immunodeficiency and cancer predisposition and which includes the symptom of elevated levels of ROS [Hosokawa et al., Free Radical Biol. Med., 2004, 37, 946-952]. However, this compound may suffer from bio-reduction in vivo.

It is also important that antioxidant compounds are water or aqueous soluble to provide good activity and bioavailability in biological systems and for ease of handling.

There is a need for new or alternative antioxidant compounds with acceptable aqueous solubility properties and improved resistance to bio-reduction in vivo.

### SUMMARY OF THE INVENTION

In a first aspect, the present invention provides a method of reducing oxidative stress in a cell comprising exposing the cell to an effective amount of a compound of formula (II):

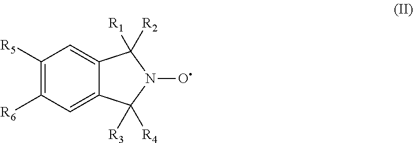

wherein
each of R₁, R₂, R₃ and R₄ are independently selected from —C₁-C₂-C₆alkenyl, —C₂-C₆alkynyl, —C₃-C₈cycloalkyl, aryl, heterocyclyl and heteroaryl wherein at least one of R₁, R₂, R₃ and R₄ is not methyl;
R₅ is selected from hydrogen, —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₆ is selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkyl SO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted; or a pharmaceutically acceptable salt thereof.

In another aspect of the present invention there is provided a method of treating or preventing a disease or disorder related to oxidative stress comprising administering an effective amount of a compound of formula (II) as defined above.

In a further aspect of the present invention there is provided a method of protecting a subject from oxidative stress upon exposure to ionising radiation comprising an effective amount of a compound of formula (II) as defined above.

In yet a further aspect of the invention, there is provided a use of a compound of formula (II) as defined above; in the manufacture of a medicament for the treatment or prevention of diseases or disorders associated with oxidative stress or in the protection of a subject from oxidative stress during treatment employing ionising radiation or radiotherapy.

In particular, in the compound of formula (II), one or more of R₁ to R₄ are selected from C₁₋₆alkyl, especially C₂-C₃alkyl, more especially ethyl, propyl or isopropyl, most especially ethyl. In some embodiments, all of R₁ to R₄ are selected from C₂₋₆alkyl, especially C₂₋₃alkyl, more especially ethyl, propyl or isopropyl, most especially ethyl. In some embodiments, R₅ is not hydrogen. In some embodiments, R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H and —C₀-C₆alkylCONH₂, especially where at least one of R₅ and R₆ is selected from —CO₂H, —NH₂, —OH, —CH₂OH, —CH₂PO₃H₂ and heterocyclyl. In some embodiments, one of R₅ and R₆ is a pyrazolyl or tetrazolyl group.

In yet a further aspect of the invention there is provided compound of formula (I):

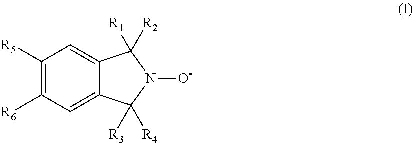

wherein
each of R₁, R₂, R₃ and R₄ are independently selected from —C₁-C₆alkyl, —C₂-C₆alkenyl, —C₂-C₆alkynyl, —C₃-C₈cycloalkyl, aryl, heterocyclyl and heteroaryl wherein at least one of R₁, R₂, R₃ and R₄ is not methyl;
R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted; or a pharmaceutically acceptable salt thereof.

In particular, in the compound of formula (I), one or more of R₁ to R₄ are selected from C₁₋₆alkyl, especially C₂-C₃alkyl, more especially ethyl, propyl or isopropyl, most especially ethyl. In some embodiments all of R₁ to R₄ are selected from C₂₋₆alkyl, especially C₂₋₃alkyl, more especially ethyl, propyl or isopropyl, most especially ethyl. In some embodiments, R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H and —C₀-C₆alkylCONH₂, especially where at least one of R₅ and R₆ is selected from —CO₂H, —NH₂, —OH, —CH₂OH, —CH₂PO₃H₂ and heterocyclyl. In some embodiments, one of R₅ and R₆ is a pyrazolyl or tetrazolyl group.

In another aspect of the invention there is provided a pharmaceutical composition comprising a compound of formula (I) or a compound of formula (II) together with a pharmaceutically acceptable carrier or excipient.

In particular embodiments, the compound of formula (II) or compound of formula (I) is a compound in which all of R₁ to R₄ are ethyl and R₅ and R₆ are both —CO₂H, —CH₂OH or —CH₂PO₃H₂, especially 1,1,3,3-tetraethyl-5,6-dicarboxylisoindoline-2-yloxyl (DCTEIO).

### DETAILED DESCRIPTION OF THE INVENTION

### Methods of Treating or Preventing Diseases or Disorders Related to Oxidative Stress

The present invention provides a method of reducing oxidative stress in a cell comprising exposing the cell to an effective amount of a compound of formula (II):

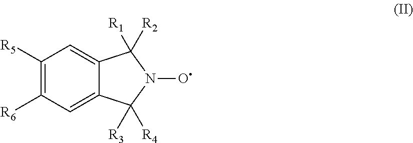

wherein
each of R₁, R₂, R₃ and R₄ are independently selected from —C₁-C₆alkyl, —C₂-C₆alkenyl, —C₂-C₆alkynyl, —C₃-C₈cycloalkyl, aryl, heterocyclyl and heteroaryl wherein at least one of R₁, R₂, R₃ and R₄ is not methyl;
R₅ is selected from hydrogen, —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₆ is selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇; and
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted; or a pharmaceutically acceptable salt thereof.

It should be understood that the cell which is treated according to a method of the present invention may be located ex vivo or in vivo. By “ex vivo” is meant that the cell has been removed from the body of a subject wherein the modulation of its activity will be initiated in vitro. For example, the cell may be a cell which is to be used as a model for studying any one or more aspects of the pathogenesis of conditions which are associated with oxidative stress or may be a cell that is sensitive to ionising radiation. In a particular embodiment, the subject cell is located in vivo.

The present invention also provides a method of treating or preventing a disease or disorder related to oxidative stress comprising administering to a subject a compound of formula (II) or a pharmaceutically acceptable salt thereof.

There is also provided a use of a compound of formula (II) or a pharmaceutically acceptable salt thereof in the manufacture of a medicament for the treatment or prevention of a disease or disorder related to oxidative stress.

Diseases or disorders related to oxidative stress are those in which there is an imbalance between the formation of pro-oxidants and the neutralisation of pro-oxidants. The pro-oxidants are reactive oxygen species (ROS) that are formed in cells such as peroxide, hydroxyl radical, nitric oxide, peroxynitrite, superoxide anion and peroxyl radicals. Such ROS in turn oxidise biological molecules in cells such as lipids and fatty acids, proteins, glycation end products and DNA. As a result, ROS have a toxic effect on cells leading to DNA damage, mitochondrial malfunction, cell membrane damage and eventually cell death.

Diseases and disorders related to oxidative stress include neurological disorders, genetic disorders, immune disorders, chronic fatigue syndrome, liver disorders, inflammatory disorders, ischemic disorders, cancer and aging. Neurological disorders related to oxidative stress include, but are not limited to, Alzheimer's disease, Parkinson's disease, multiple sclerosis, amyotrophic lateral sclerosis (ALS), neurofibromatosis, Duschenne Muscular Dystrophy and dementia including AIDS related dementia. Genetic disorders related to oxidative stress include, but are not limited to, ataxia telangiectasia (A-T), ataxia telangiectasia-like disorder (ATLD), Nijmegen breakage syndrome (NBS), ataxia oculomotor apraxia type 1 or type 2, Goucher disease, Hartnup disease, Nieman-Pick disease, Refsum disease, Friedrich's Ataxia, Cockayne Syndrome, motor neurone disease and neurofibromatosis. Immune disorders related to oxidative stress include HIV. Liver disorders associated with oxidative stress include hepatitis. Inflammatory disorders related to oxidative stress include rheumatoid arthritis. Ischemic disorders related to oxidative stress include, but are not limited to, stroke, myocardial infarction, cardiac ischemia and reperfusion injury. Many cancers, including tumor related cancers, such as prostate cancer, breast cancer, lung cancer, brain tumors, liver cancer, bone cancer, kidney cancer, stomach cancer and colon cancer, have been associated with oxidative stress. Antioxidants may have direct toxicity on cancer cells or may act indirectly through interfering with stages of tumorigenesis and cancer progression such as cancer cell migration, invasion and adhesion.

In some embodiments of the invention, the compounds of formula (II) are used to treat or prevent A-T, ATLD, NBS, ataxia oculomotor apraxia type 1 or type 2, Goucher disease, Hartnup disease, Nieman-pick disease, Refsum disease, Friedrich's Ataxia, Cockayne Syndrome or neurofibromatosis, especially A-T. A-T is a genetic disorder characterised by neurodegeneration, immunodeficiency and a predisposition to cancer which also has the symptom of elevated ROS. Those suffering from A-T have hypersensitivity to ionising radiation and therefore, treatment of cancers in these individuals with radiotherapy and chemotherapy must be approached with caution. The compounds of formula (II) provide protection from the effects of ionising radiation in those subjects suffering from A-T or carriers of a defective A-T gene with normal phenotype.

In a further aspect of the present invention, there is provided a method of protecting a subject from oxidative stress upon exposure to ionising radiation comprising administering to the subject an effective amount of a compound of formula (II) or a pharmaceutically acceptable salt thereof.

This method is suitable for protection against oxidative stress during or subsequent to accidental or unplanned exposure to ionising radiation or planned exposure to ionising radiation. For example, a person at risk of exposure to ionising radiation during warfare or at a nuclear power facility or other facility that may under normal circumstances produce controlled ionising radiation may benefit from administration of a compound of formula (II). Similarly, a person recently exposed to ionising radiation after explosion of a nuclear bomb or an accident at a nuclear facility may benefit from administration of a compound of formula (II) to minimise oxidative stress resulting from exposure in the ionising radiation. Furthermore, a person requiring therapy with ionising radiation or radiotherapy, for example in cancer therapy, may benefit from administration of a compound of formula (II). The compound of formula (II) may be administered prophylactically before exposure; or may be administered simultaneously with or immediately after exposure to ionising radiation such as described above.

This method may be particularly useful for subjects with A-T or carriers of a defective A-T gene with normal phenotype and also in the general population of those requiring therapy with ionising radiation or radiotherapy. The administration of compounds of formula (II) may also provide protection against or reduction in side effects associated with therapy with ionising radiation such as hair loss. In particular embodiments, the compound of formula (II) is administered prior to and/or simultaneously with ionising radiation or radiotherapy.

Reference herein to “protecting” and “protection” is to be considered in its broadest context. The term “protection” does not necessarily imply that a subject suffers no effects of oxidative stress or no side effects associated with exposure to or therapy with ionising radiation or radiotherapy. Accordingly, prevention includes amelioration of the effects of a oxidative stress or side effects or reducing the severity or delaying the onset of the effects.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 0.5 g per kg of body weight per dosage. The dosage is preferably in the range of 1 μg to 0.5 g per kg of body weight per dosage, such as is in the range of 1 mg to 0.5 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation.

The terms “subject”, “individual” and “patient” are used herein interchangeably and may be any subject, individual or patient that is undergoing or likely to undergo oxidative stress. The subject, individual or patient may be a mammal. Suitable mammals include humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). In some embodiments, the mammal is human or a laboratory test animal, especially a human.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis includes amelioration of the symptoms of a particular condition or preventing or otherwise reducing the risk of developing a particular condition. The term “prophylaxis” may be considered as delaying the onset or reducing severity of a particular condition after delayed onset. “Treatment” may reduce the severity of an existing condition.

The present invention further contemplates a combination of therapies, such as the administration of the compounds of the invention or pharmaceutically acceptable salts or prodrugs thereof together with the subjection of the subject to other agents or procedures which are useful in the treatment of diseases and conditions associated with oxidative stress. For example, the compounds of the present invention may be administered in combination with other agents suitable for treating or preventing neurological disorders, genetic disorders, immune disorders, chronic fatigue syndrome, liver disorders, inflammatory disorders, cancer and aging, or may be used with other treatments such as radiotherapy. Suitable agents comprise antioxidants, including but not limited to, N-acetylcysteine, lipoic acid, Tempol, Trolox and Edaravone; electron transport chain blockers including but not limited to, Rotenone and antimycin-A; immunosuppressants; metabolic inhibitors including but not limited to, 2-deoxyglucose; reverse transcriptase inhibitors and other antiviral agents and chemotherapeutic drugs including but not limited to, carboplatin, doxorubicin, paclitaxel, docetaxel and other taxol and taxane drugs.

The term “in combination with” refers to administration of the compounds of formula (II) with another agent such that they are both biologically active, at least partially, at the same time. The compounds of formula (II) and the other agent may, be administered in the same composition or in separate compositions simultaneously or sequentially.

In some embodiments, the compounds of formula (II) are compounds of formula (I) described below.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### Compounds of the Invention

The present invention relates to compounds of formula (I):

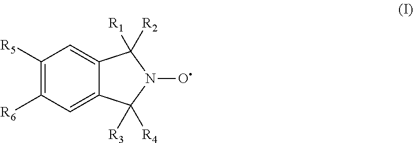

wherein
each of R₁, R₂, R₃ and R₄ are independently selected from —C₁-C₆alkyl, —C₂-C₆alkenyl, —C₂-C₆alkynyl, —C₃-C₈cycloalkyl, aryl, heterocyclyl and heteroaryl wherein at least one of R₁, R₂, R₃ and R₄ is not methyl;
R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇; and
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted; or a pharmaceutically acceptable salt thereof.

The definitions below apply to compounds of formula (I) and compounds of formula (II).

As used herein, the term “alkyl” refers to a straight chain or branched saturated hydrocarbon group having 1 to 10 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement.

Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

As used herein, the term “alkenyl” refers to a straight-chain or branched hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl and decenyl.

As used herein, the term “alkynyl” refers to a straight-chain or branched hydrocarbon group having one or more triple bonds between carbon atoms and having 2 to 10 carbon atoms. Where appropriate, the alkynyl group may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to, ethynyl, propynyl, butynyl, pentynyl, hexynyl, octynyl, nonynyl and decynyl.

As used herein, the term “cycloalkyl” refers to a saturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentanyl, cyclohexanyl, cycloheptanyl and cyclooctanyl.

The terms “alkyloxy” or “alkoxy”, “alkenyloxy” and “alkynyloxy” as used herein represent an alkyl, alkenyl or alkynyl group as defined above attached through an oxygen bridge. Examples of suitable alkyloxy, alkenyloxy and alkynyloxy groups include, but are not limited to, methoxy, ethoxy, n-propyloxy, n-butyloxy, n-pentyloxy, n-hexyloxy, ethenyloxy, propenyloxy, butenyloxy, pentenyloxy, hexenyloxy, ethynyloxy, propynyloxy, butynyloxy, pentynyloxy and hexynyloxy.

The terms “alkylthio”, “alkenylthio” and “alkynylthio” as used herein represent an alkyl, alkenyl or alkynyl group as defined above attached through a sulfur bridge. Examples of suitable alkylthio, alkenylthio and alkynylthio include, but are not limited to, methylthio, ethylthio, propylthio, butylthio, pentylthio, hexylthio, ethenylthio, propenylthio, butenylthio, pentenylthio, hexenylthio, ethynylthio, propynylthio, butynylthio, pentynylthio and hexynylthio.

The term “acyl” used herein refers to an alkoyl or aroyl group as defined by (C═O)R^{a} where suitable R^{a} groups include, but are not limited to, C₁₋₆alkyl, C₁₋₆alkenyl, C₁₋₆alkynyl, C₃₋₈cycloalkyl, aryl, heterocyclyl, heteroaryl, C₁₋₆alkylcycloalkyl, C₁₋₆alkylheterocyclyl, C₁₋₆alkylheteroaryl, C₁₋₆alkoxyalkyl, C₁₋₆alkylthioalkyl, C₁₋₆alkylthioaryl, C₁₋₆alkoxyaryl and the like.

As used herein, the term “aryl” is intended to mean any stable, monocyclic or bicyclic carbon ring of up to 7 atoms in each ring, wherein at least one ring is aromatic. Examples of such aryl groups include, but are not limited to, phenyl, naphthyl, tetrahydronaphthyl, indanyl, biphenyl and binaphthyl.

As used herein, the term “halogen” or “halo” refers to fluorine (fluoro), chlorine (chloro), bromine (bromo) and iodine (iodo).

The term “heterocyclic” or “heterocyclyl” as used herein, refers to a cyclic hydrocarbon in which one to four carbon atoms have been replaced by heteroatoms independently selected from the group consisting of N,N(R), S, S(O), S(O)₂ and O. A heterocyclic ring may be saturated or unsaturated. Examples of suitable heterocyclyl groups include tetrahydrofuranyl, tetrahydrothiophenyl, pyrrolidinyl, pyrrolinyl, pyranyl, piperidinyl, pyrazolinyl, pyrazolinonyl pyrazolidinyl, piperazinyl, imidazolidinyl, dithiolyl, oxathiolyl, dioxanyl, dioxalanyl, dioxinyl, thiazolinyl, dioxazolyl, oxazolonyl, oxathiozolyl, oxazinyl, oxathiazinyl, 2-pyronyl, 4-pyronyl, morpholino, thiomorpholinyl, dithianyl, trithianyl, guanine, thymine, uracil, cytosine, guanosine, 5-methyluridine, thymidine, uridine, cytadine, deoxyguanosine, deoxyuridine and deoxycytidine and pyranose and furanose sugars.

The term “heteroaryl” as used herein, represents a stable monocyclic or bicyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, quinazolinyl, pyrazolyl, indolyl, benzotriazolyl, furanyl, thienyl, thiophenyl, benzothienyl, benzofuranyl, benzodioxane, benzodioxin, quinolinyl, isoquinolinyl, oxazolyl, isoxazolyl, imidazolyl, pyrazinyl, pyridazinyl, pyridinyl, pyrimidinyl, pyrrolyl, tetrahydroquinolinyl, thiazolyl, isothiazolyl, 1,2,4-triazolyl, 1,2,4-oxadiazolyl, 1,2,4-thiadiazolyl, 1,3,5-triazinyl, 1,2,4-triazinyl, 1,2,4,5-tetrazinyl, tetrazolyl, adenine, adenosine and deoxyadenosine. Particular heteroaryl groups have 5- or 6-membered rings, such as pyrazolyl, furanyl, thienyl, oxazolyl, isoxazolyl, imidazolyl, pyrazinyl, pyridazinyl, pyridinyl, pyrimidinyl, pyrrolyl, thiazolyl, isothiazolyl, 1,2,4-triazolyl, tetrazolyl, 1,2,4-oxadiazolyl and 1,2,4-thiadiazolyl.

Each alkyl, alkenyl, alkynyl, cycloalkyl, aryl, heterocyclyl and heteroaryl, whether an individual entity or as part of a larger entity may be optionally substituted with one or more optional substituents selected from the group consisting of C₁₋₆alkyl, C₂₋₆alkenyl, C₂₋₆alkynyl, C₃₋₆cycloalkyl, C₁₋₆alkyloxy-, C₂₋₆alkenyloxy-, C₂₋₆alkynyloxy-, C₃₋₆cycloalkoxy-, C₁₋₆alkylthio-, C₂₋₆alkenylthio-, C₂₋₆alkynylthio-, C₂₋₆cycloalkylthio-, hydroxy, —SH, —CO₂H, —CO₂C₁₋₆alkyl, —CON(R₈)₂, C₂₋₆acyl-, C₂₋₆acyloxy-, C₂₋₆alkylSO₂—, C₂₋₆alkenylSO₂—, C₂₋₆alkynylSO₂—, —NH₂, —NH(C₁₋₆alkyl), —N(C₁₋₆alkyl)₂, —NH(phenyl), —N(phenyl)₂, —NH(acyl), —N(acyl)(phenyl), —N═NHC(O)NH₂, —NHSO₂R₈, —SO₂N(R₈)₂, —C(R₉)₃, —OC(R₉)₃, —SC(R₉)₃, —CN, —NO₂ and halogen, wherein each R₉ is independently selected from hydrogen and halogen and each R₈ is independently selected from hydrogen, C₁₋₆alkyl, phenyl, cycloalkyl or the two R₈ taken together with the nitrogen to which they are attached can form a heterocyclyl or heteroaryl ring. Examples of suitable substituents include, but are not limited to, methyl, ethyl, propyl, isopropyl, butyl, sec-butyl, tert-butyl, vinyl, methoxy, ethoxy, propoxy, isopropoxy, butoxy, methylthio, ethylthio, propylthio, isopropylthio, butylthio, hydroxy, hydroxymethyl, hydroxyethyl, hydroxypropyl, hydroxybutyl, fluoro, chloro, bromo, iodo, cyano, nitro, CO₂H, CO₂CH₃, CO₂CH₂CH₃, trifluoromethyl, trifluoromethoxy, trifluoromethylthio, acetyl, morpholino, amino, methylamino and dimethylamino.

The compounds of the invention may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; diallyl sulfates like dimethyl and diethyl sulfate; and others.

The compounds and salts of the invention may be presented in the form of a prodrug. The term “prodrug” is used in its broadest sense and encompasses those derivatives that are converted in vivo to the compounds of the invention. Such derivatives would readily occur to those skilled in the art, and include esters and amides (including amino acid esters, amides and conjugates), N-α-acyloxy amides. A prodrug may include modifications to one or more of the functional groups of a compound of the invention.

The term “prodrug” also encompasses the combination of lipids with the compounds of the invention. The presence of lipids may assist in the translocation of the compounds across a cellular membrane and into a cell cytoplasm or nucleus. Suitable lipids include fatty acids which may be linked to the compound by formation of a fatty acid ester. Particular fatty acids include, but are not limited to, lauric acid, caproic acid, palmitic acid and myristic acid.

The phrase “a derivative which is capable of being converted in vivo” as used in relation to another functional group includes all those functional groups or derivatives which upon administration into a mammal may be converted into the stated functional group. Those skilled in the art may readily determine whether a group may be capable of being converted in vivo to another functional group using routine enzymatic or animal studies.

It will also be recognised that compounds of the invention may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. The compounds of the invention may also exist as geometric isomers. The invention also relates to compounds in substantially pure cis (Z) or trans (E) or mixtures thereof.

In some embodiments, the compound of formula (I) is a compound of formula (IA):

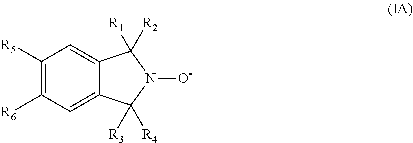

wherein
each of R₁, R₂, R₃ and R₄ are independently selected from —C₂-C₆alkyl, —C₂-C₆alkenyl, —C₂-C₆alkynyl, —C₃-C₈cycloalkyl, aryl, heterocyclyl and heteroaryl;
R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇; and
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted; or a pharmaceutically acceptable salt thereof.

In particular embodiments of the compounds of formula (I), one or more of the following applies:

R₁, R₂, R₃ and R₄ are the same or different, provided that at least one of R₁ to R₄ is not methyl, and are selected from C₁-C₆alkyl, especially C₁-C₃alkyl, more especially methyl, ethyl, propyl or isopropyl, more especially where one or more of R₁, R₂, R₃ and R₄ are ethyl, propyl or isopropyl, or where all of R₁, R₂, R₃ and R₄ are selected from C₂-C₆alkyl, especially C₂ or C₃alkyl, more especially ethyl, propyl or isopropyl, most especially where all of R₁, R₂, R₃ and R₄ are ethyl.
R₅ is —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, especially where R₅ is absent or is —C₀-C₃alkylCO₂H, —C₀-C₃alkylNH₂, —C₀-C₃alkylOH, —C₀-C₃alkylPO₃H₂, —C₀-C₃alkylhalo, —C₀-C₃alkylNO₂, —C₀-C₃alkylCN, —C₀-C₃alkylheterocyclyl, —C₀-C₃alkylheteroaryl, —C₀-C₃alkylSO₃H, —C₀-C₃alkylCONH₂, more especially where R₅ is —CO₂H, —NH₂, —OH, —CH₂OH, —CH₂PO₃H₂ or heterocyclyl, especially where heterocyclyl is an optionally substituted pyrazolyl or tetrazolyl group, more especially an optionally substituted 5-oxo-pyrazolyl group, most especially a 3-methyl-5-oxo-pyrazolyl group.
R₆ is —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, especially where R₆ is —C₀-C₃alkylCO₂H, —C₀-C₃alkylNH₂, —C₀-C₃alkylOH, —C₀-C₃alkylPO₃H₂, —C₀-C₃alkylhalo, —C₀-C₃alkylNO₂, —C₀-C₃alkylCN, —C₀-C₃alkylheterocyclyl, —C₀-C₃alkylheteroaryl, —C₀-C₃alkylSO₃H, —C₀-C₃alkylCONH₂, more especially where R₆ is —CO₂H, —NH₂, —OH, —CH₂OH, —CH₂PO₃H₂ or heterocyclyl, especially where heterocyclyl is an optionally substituted pyrazolyl or tetrazolyl group, more especially an optionally substituted 5-oxo-pyrazolyl group, most especially a 3-methyl-5-oxo-pyrazolyl group.
R₇ is selected from —C₁-C₆alkyl, —C₂-C₆alkenyl, —C₂-C₆alkynyl, —C₃-C₈cycloalkyl, aryl, heterocyclyl and heteroaryl.

In some embodiments, the compounds of the invention are:

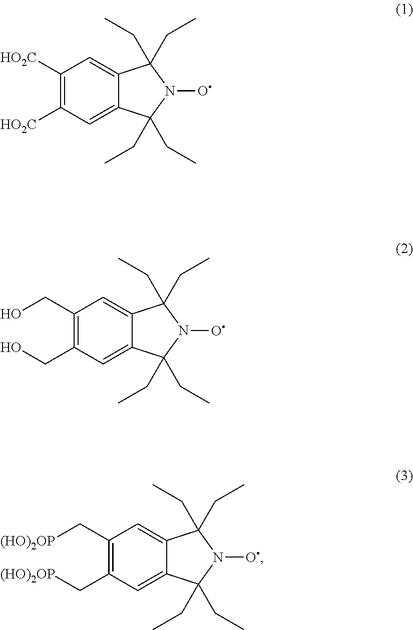

especially compound (1) (DCTEIO).

A particular compound of formula (II) is:

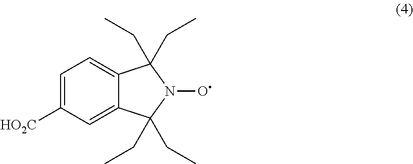

The compounds of formula (I) and formula (II) can be synthesised from known starting materials, using known methods.

For example, 1,1,3,3-tetrasubstituted isoindoline compounds may be purchased if available or may be prepared by Grignard reaction as shown in Scheme 1 (where R is alkyl or alkenyl, such as methyl, ethyl, propyl, isopropyl or ethenyl and P is a protecting group):

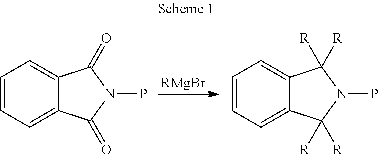

The tetrasubstituted isoindoline, after protection may be brominated (Scheme 2, where R and P are as defined in Scheme 1), for example using bromine and aluminium trichloride (AlCl₃) in dichloromethane or dibrominated (Scheme 3, where R and P are as defined in Scheme 1), for example with bromine, aluminium trichloride (AlCl₃) and pyridine in chloroform.

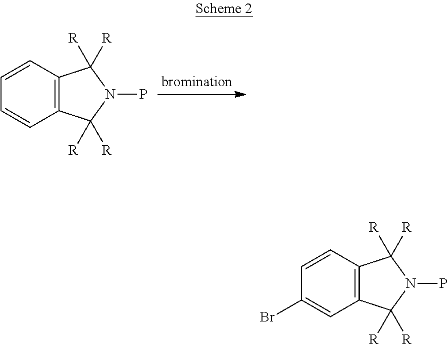

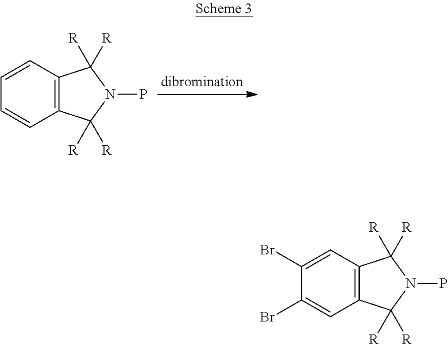

The bromine groups may be replaced with carboxylic acid groups or other substituents in a number of ways. For example, lithiation or dilithiation (nBuLi in THF) followed by treatment with CO₂, cyanation or dicyanation (potassium hexacyanoferrate, K₄[Fe(CN₆)], copper iodide (C(O)I) and n-butylimidazole (nBulmi) in toluene, or palladium(0) catalysed coupling with zinc chloride) followed by basic hydrolysis (KOH, H₂O, ethanol) or oxidation of aromatic methyl groups (KMnO₄ in pyridine/H₂O).

The amino group of the isoindoline may be protected during substitution of the 1, 3, 5 and/or 6 positions if required by the reaction conditions used. Suitable protecting groups may be found in Greene and Wuts, “Protective Groups in Organic Synthesis”, 3^{rd} Edition, Wiley Interscience, 1999. For example, the amino group may be protected by a benzyl group. After the substitution reactions have occurred, the protecting group may be deprotected. The free amino group may then be oxidised to form the nitroxide radical. Suitable conditions for oxidising the amino group include treatment of the amino group with hydrogen peroxide, mild base such as sodium bicarbonate (NaHCO₃) and disodium wolframate dihydrate (Na₂WO₄.2H₂O) in methanol or in some cases, meta-chloroperbenzoic acid (mCPBA) in dichloromethane.

Alternatively, the nitroxide group may be introduced into the isoindoline early in the synthetic pathway as described above and then reduced to a hydroxylamine and protected, for example, with an acetate group. Other suitable protecting groups may be found in Greene and Wuts, ibid. After appropriate substitution or derivatisation of substitutents, the nitroxide can be regenerated from the protected hydroxylamine, for example with mild base such as lithium hydroxide (LiOH) in water.

It is also possible to introduce other substituents in the 5 and/or 6-position of the isoindoline compound or to derivatise substituents in those positions. For example, 5 and/or 6-carboxy substituents can be reduced to provide hydroxymethyl substituents as shown in Scheme 4 (R and P are as defined in Scheme 1). A suitable reducing agent is the selective reducing agent lithium aluminium chloride (LiAlH₄) in ether.

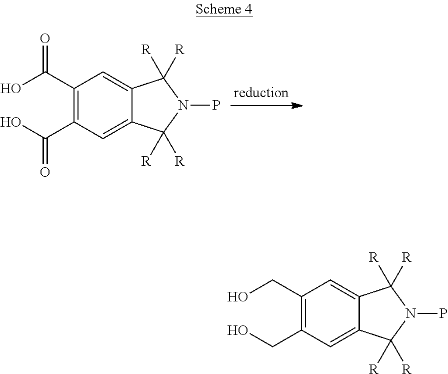

The dihydroxymethyl substituted isoindoline shown in Scheme 4 may be further derivatised to prepare a dibromomethyl substituent by bromination, for example with phosphorus tribromide in dichloromethane and then treated with triethyl phosphite to give a diphosphonic acid after deprotection of the phosphonate ethyl groups as shown in Scheme 5 (R and P are as defined in Scheme 1).

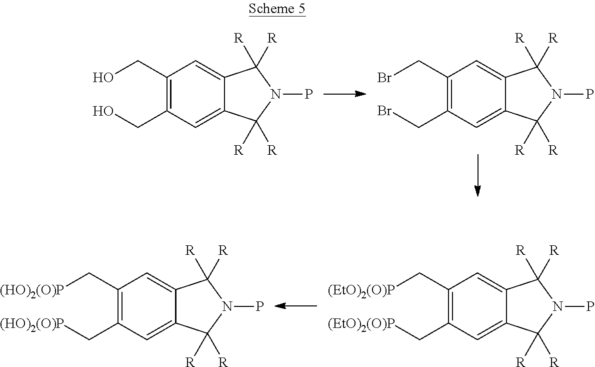

Other substituents may be introduced into the 5- or 6-position of the isoindoline by substitution on the benzene ring. One example is the nitration of 5-bromo-1,1,3,3-tetraalkylisoindoline under standard nitration conditions of HNO₃ and H₂SO₄ to provide 5-bromo-6-nitro-1,1,3,3-tetraalkylisoindoline. The nitro group may then be reduced to provide an amino substituent. The bromo group may then be substituted with another substituent such as a cyano group as described above to enable the formation of a carboxylic acid or with other groups such as an α,β-unsaturated ester thereby providing a method of achieving longer chain substituents as shown in Scheme 6 (R is as defined in Scheme 1). Methods of aromatic substitution are known in the art.

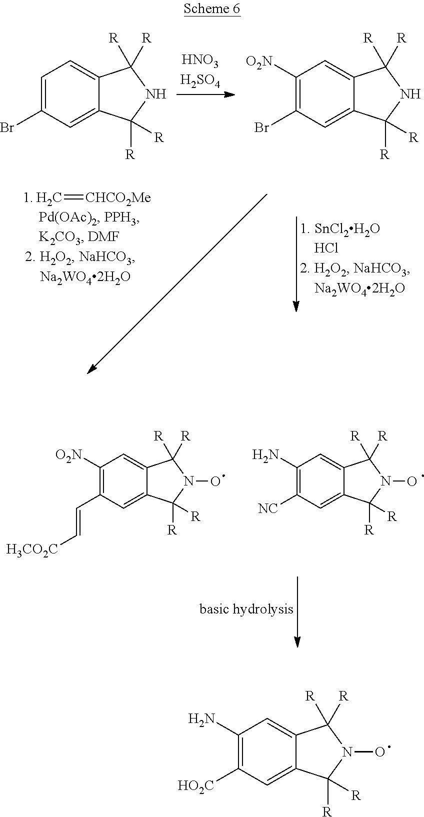

### Compositions of the Invention

While it is possible that, for use in therapy, a compound of formula (I) or formula (II) may be administered as a neat chemical, it is preferable to present the active ingredient as a pharmaceutical composition.

Therefore in another aspect of the invention, there is provided a pharmaceutical composition comprising a compound of formula (I) or formula (II) or a pharmaceutically acceptable salt thereof and at least one pharmaceutically acceptable carrier.

The carrier(s) must be “acceptable” in the sense of being compatible with the other ingredients of the composition and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The compounds of the invention, together with a conventional adjuvant, carrier, excipient, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to two hundred (200) milligrams, per tablet, are accordingly suitable representative unit dosage forms. The compounds of the present invention can be administered in a wide variety of oral and parenteral dosage forms. It will be obvious to those skilled in the art that the following dosage forms may comprise, as the active component, either a compound of the invention or a pharmaceutically acceptable salt or derivative of the compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules.

A solid carrier can be one or more substances which may also act as diluents, flavouring agents, solubilisers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds according to the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP).

Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 1 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The invention will now be described with reference to the following Examples which illustrate some preferred aspects of the present invention. However, it is to be understood that the particularity of the following description of the invention is not to supersede the generality of the preceding description of the invention.

### BRIEF DESCRIPTION OF THE FIGURES

FIG. 1 provides a schematic diagram of the treatment of the cells in the cell survival assay.

FIG. 2 provides a graphical representation of (A) the survival of KT cells in the presence or absence of compound (1) when the cells are irradiated and (B) the growth of KT cells in the presence of compound (1).

FIG. 3 provides a graphical representation of (A) the survival of KT cells in the presence or absence of compound (2) when the cells are irradiated and (B) the growth of KT cells in the presence of compound (2).

FIG. 4 provides a graphical representation of (A) the survival of KT cells in the presence or absence of compound (3) when the cells are irradiated and (B) the growth of KT cells in the presence of compound (3).

FIG. 5 provides Electron Spin Resonance spectra of CMTIO (FIG. 5A) and compound (1) (DCTEIO).

### EXAMPLES

### General Methods

All air-sensitive reactions were carried out under an atmosphere of ultra-high purity argon. Ether and toluene were dried by storage over sodium wire. Tetrahydrofuran (THF) was freshly distilled from sodium benzophenone ketal and dichloromethane (DCM) freshly distilled from calcium hydride. Triethylamine and pyridine were dried by storage over potassium hydroxide. Crystalline K₄[Fe(CN)₆].3H₂O was ground to a fine powder and then dried at 80° C. at 0.5 Torr for 10 hours. 2-Benzyl-1,1,3,3-tetraethylisoindoline was prepared by literature procedures provided by Heidenbluth and Scheffler (Journal fuer Praktische Chemie, 1964, 23(1-2), 59-79). 4,5-Dibromophthalic anhydride was prepared by the literature method of Gould et al. (J. Chem. Soc., Perkin Trans 1, 1980, 8, 1834-1840). 4,5-Dimethylphthalic anhydride was prepared by the methods of Gould et al. (ibid), Bailey et al. (J. Am. Chem. Soc., 1954, 76, 2251-2254) or Toyooka et al. (WO2004048332). All other reagents were purchased from commercial suppliers and used without further purification.

¹H and ¹³C NMR spectra were recorded on a Bruker Avance 400 spectrometer and referenced to the relevant solvent peak. Low and high resolution mass spectra were recorded at the Australian National University (ANU) using either a Micromass autospec double focusing magnetic sector mass spectrometer (EI⁺ spectra) or a Bruker Apex 3 fourier transform ion cyclotron resonance mass spectrometer with a 4.7 T magnet (ESI⁺ spectra). Formulations were calculated in the elemental analysis programs of Mass Lynx 4.0 or Micromass Opus 3.6. Fourier transform infrared (FTIR) spectra were recorded on a Nicolet 870 Nexus Fourier Transform Infrared Spectrometer equipped with a DTGS TEC detector and an ATR objective. Elemental analyses were carried out by the University of Queensland Microanalytical Service. Melting points were measured on a GallenKamp Variable Temperature Apparatus by the capillary method and are uncorrected.

### Example 1

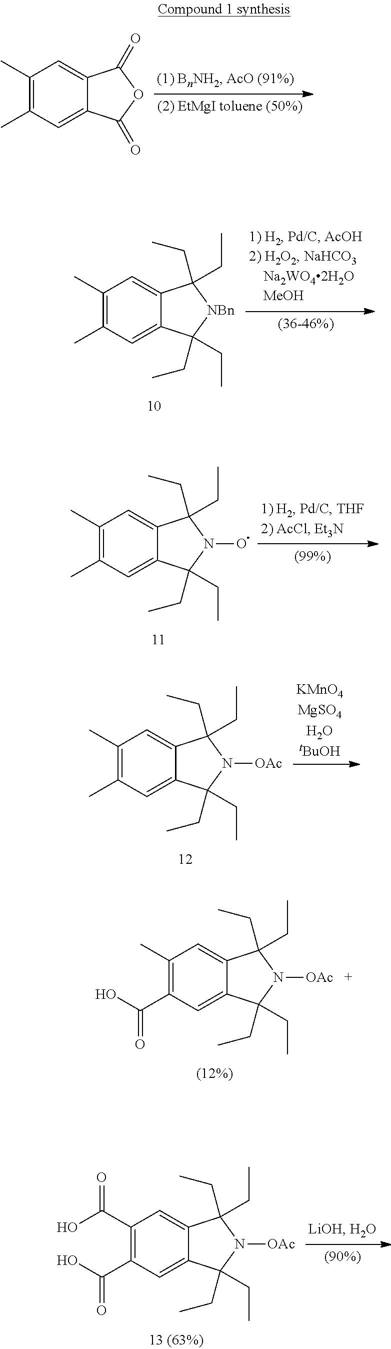

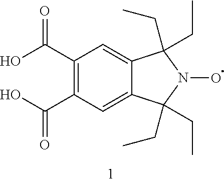

2-Benzyl-5,6-dimethyl-1,1,3,3-tetramethylisoindoline (10): A suspension of 5,6-dimethylphthalic anhydride (7.8 g, 44.3 mmol, 1.0 equiv) in acetic acid (50 mL) was treated with benzylamine (6.28 mL, 57.6 mmol, 1.30 equiv), warmed to 120° C. and stirred at this temperature for 1.5 h. The mixture was poured into ice/H₂O mixture (100 mL) and filtered. The residue was recrystallised from ethanol to yield 10.7 g of 2-benzyl-5,6-dimethylphthalamide as colourless, voluminous crystals (40.3 mmol, 91%) M.p. 138-140° C. ¹H NMR (CDCl₃, 400 MHz): δ=2.41 (s, 6H, CH₃), 4.83 (s, 2H, CH₂), 7.23-7.35 (m, 3H, Ar—H), 7.40-7.45 (m, 2H, Ar—H) 7.61 (s, 2H, Ar—H) ppm. ¹³C NMR (CDCl₃, 100 MHz, add. DEPT): δ=20.6 (+, CH₃), 41.5 (−, CH₂), 124.3, 127.7, 128.5, 128.6 (+, 7C, Ar—C, 130.1, 136.6, 143.7 (C_{quat}, 5C, Ar—C) ppm. MS (EI): m/z (%)=265 (100) [M⁺], 247 (78), 236 (58), 222 (67), 133 (59), 104 (67), 91 (44) [C₇H₇⁺], 77 (42) [C₆H₅⁺]. HRMS (EI): m/z: calcd. for C₁₇H₁₅NO₃ [M⁺]: 265.1103; found 265.1102. C₁₇H₁₅NO₂ (265.31): calcd. C, 76.96, H, 5.70, N, 5.28; found C, 76.87; H, 5.56; N, 5.26. A solution of 2-benzyl-5,6-dimethylphthalamide (7.00 g, 26.38 mmol, 1.00 equiv.) in anhydrous toluene (62 mL) was treated with ethyl magnesium iodide [freshly prepared from ethyl iodide (12.66 mL, 158.29 mmol) and magnesium turnings (7.70 g, 316.59 mmol) in Et₂O (62 mL)]. The Et₂O was distilled off via Dean-Stark. The reaction mixture was heated to reflux, stirred for 3 h and then concentrated to about half of its volume. Hexane (4×100 mL) was added, the mixture was filtered through Celite and washed thoroughly with extra hexane (100 mL). The filtrate was passed through a column of basic alumina and concentrated in vacuo to give 4.7 g of 10 as a colourless oil which solidified when kept at ambient temperature (13.46 mmol, 51%). M.p. 98-100° C. ¹H-NMR (CDCl₃, 400 MHz): δ=0.80 (t, 12H, ³J=7.0Hz, CH₂CH₃), 1.45-1.65 (m, 4H, CH₂CH₃), 1.85-2.00 (m, 4H, CH₂CH₃), 2.30 (s, 6 H, CH₃), 4.01 (s, 2H, CH₂), 6.84 (s, 2H, Ph-H), 7.22-7.40 (m, 3H, Ph-H), 7.45-7.55 (m, 2H, Ph-H). ¹³C-NMR (CDCl₃, 101 MHz, add. DEPT): δ=9.7 (+, CH₂CH₃), 20.1 (−, CH₂CH₃), 30.3 (+, PhCH₃), 46.8 (C_{quat}, CCH₃), 71.1 (−, NCH₂), 124.5, 127.8, 129.3, 133.7 (+, 7 C, Ph-C), 126.5, 142.3, 142.6 (C_{quat}, 5 C, Ph-C). MS (EI): m/z (%)=348 (3) [M⁻-H], 320 (100), 236 (58), 91 (47) [C₇H₇⁺]. HRMS (EI): calcd. for C₂₅H₃₄N[M⁻-H]: 348.2691, found 348.2690. C₂₅H₃₅N (349.56): calcd. C, 85.90; H, 10.09; N, 4.01; found: C, 85.76, H, 10.36, N, 4.00.

1,1,3,3-Tetraethyl-5,6-dimethylisoindoline-2-yloxyl (11): The isoindoline derivative 10 (2.15 g, 6.15 mmol, 1.00 equiv.) was dissolved in AcOH (25 mL). Ar was bubbled over the reaction mixture for 10 min. Palladium (328 mg, 308 μmol, 10% on charcoal, 5 mol %) was added and Ar was again bubbled over the mixture for 10 min. The reaction mixture was set under an atmosphere of hydrogen and shaken at 50 psi in a Parr apparatus for 3 h. Ar was bubbled over the mixture for 10 min. The mixture was filtered through Celite and concentrated under reduced pressure. The residue was filtered through SiO₂ (20 g, hexane/EtOAc 5:1) and evaporated in vacuo to give 1,1,3,3-tetraethyl-5,6-dimethylisoindoline. ¹H-NMR (CDCl₃, 400 MHz): δ=0.90 (t, 12H, ³J=7.0Hz, CH₂CH₃), 1.55-1.80 (m, 8H, CH₂CH₃), 2.29 (s, 6H, PhCH₃), 6.86 (s, 2H, Ph-H). ¹³C-NMR (CDCl₃, 100 MHz)=9.02 (CH₂CH₃), 20.07 (CH₂CH₃), 33.80 (PhCH₃), 68.06 (CCH₂), 123.56, 134.72, 145.26 (3 C, Ph-C). The residue was dissolved in MeOH (20 mL), treated with NaHCO₃ (775 mg, 9.23 mmol, 1.50 equiv.), Na₂WO₄.2H₂O (144 mg, 461 μmol, 7.5 mol %) and then hydrogen peroxide (3.17 mL, 30.8 mmol, 30% in H₂O, 5.00 equiv.) and the reaction mixture was stirred for 1 d. A second portion of NaHCO₃ (775 mg, 9.23 mmol, 1.50 equiv.), Na₂WO₄.2H₂O (144 mg, 461 μmol, 7.5 mol %) and hydrogen peroxide (3.17 mL, 30.8 mmol, 30% in H₂O, 5.00 equiv.) was added and stirring was continued for an additional 2 d. The reaction mixture was concentrated to half of its volume, acidified by careful addition of 2 M aq. H₂SO₄ sol. (20 mL) and extracted with EtOAc (3×20 mL). The combined organic layers were dried over MgSO₄ and evaporated under reduced pressure. The residue was filtered through SiO₂ (40 g, hexane/EtOAc 5:1) to give 610 mg of 11 as an orange solid (2.22 mmol, 36%). Recrystallisation from EtOAc yielded 11 as orange crystals, m. p. 127-129° C. On a 2.86 mmol scale, a yield of 46% was obtained. MS (EI): m/z (%)=274 (35) [M⁺], 246 (90), 230 (48), 200 (52). HRMS (EI): m/z: calcd. for C₁₈H₂₈NO[M⁺]: 274.2171, found 274.2174. C₁₈H₂₈NO (274.43): calcd. C, 78.78; H, 10.28; N, 5.10; found: C, 78.68; H, 10.34; N, 5.08.

N-Acetyloxy-1,1,3,3-tetraethyl-5,6-dimethylisoindoline (12): A solution of the nitroxide 11 (592 mg, 2.16 mmol, 1.00 equiv.) in THF (10 mL) was treated with palladium (57.5 mg, 54.0 μmol, 10% on charcoal, 2.5 mol %) and stirred under an atmosphere of H₂ for 15 min. The reaction mixture was cooled to 0° C., NEt₃ (602 μL 4.32 mmol, 2.00 equiv.) and AcCl (383 μL, 5.38 mmol) were added and the mixture was stirred at 0° C. for 20 min. The cooling bath was removed and stirring was continued for an additional 1 h. Ar was bubbled over the mixture for 10 min. The reaction mixture was filtered through Celite and concentrated in vacuo. The residue was taken up in H₂O (15 mL) and extracted with EtOAc (3×10 mL). The combined organic extracts were dried over MgSO₄ and evaporated under reduced pressure. The residue was purified by column chromatography (30 g SiO₂, hexane/EtOAc 10:1) to give 676 mg of the product 12 as a pale yellow, viscous oil which solidified within a day when kept at ambient temperature (2.13 mmol, 99%). M. p. 80-82° C. ¹H-NMR (CDCl₃, 400 MHz): δ=0.75-0.85 (m, 6H, CH₂CH₃), 0.92-1.02 (m, 6H, CH₂CH₃), 1.60-1.70, 1.70-1.80, 1.80-1.92, 1.92-2.05 (m, 8H, CH₂CH₃), 2.13 (s, 3H, CH₃CO), 2.30 (s, 6H, PhCH₃), 6.84 (s, 2H, Ph-H). ¹³C-NMR (CDCl₃, 100 MHz, add. DEPT): δ=8.62, 9.47 (+, CH₂CH₃), 19.44, 20.05 20.08 (+, CH₃CO, PhCH₃), 28.88, 30.35 (−, CH₂CH₃), 63.15 (C_{quat}, CCH₂), 124.54 (+, Ph-C), 134.90, 139.23, (C_{quat}, Ph-C), 170.60 (C_{quat}, C═O). MS (EI): m/z (%)=316 (44) [M⁻-H], 288 (94), 246 (100), 228 (72), 200 (71). HRMS (EI): m/z: calcd. for C₂₀H₃₀NO₂[M⁻-H]: 316.2277, found 316.2280. C₂₀H₃₁NO₂ (317.47): calcd. C, 75.67; H, 9.84; N, 4.41; found: C, 75.59; H, 10.13; N, 4.38.

N-Acetyloxy-1,1,3,3-tetraethyl-5,6-dicarboxylsoindoline (13): A solution of the dimethylaryl derivative 12 (458 mg, 1.44 mmol, 1.00 equiv.) in tBuOH (10 mL) was warmed to 40° C. The mixture was treated with MgSO₄ (177 mg, 720 mol, 0.50 equiv.) and 0.4 M aq. KMnO₄-sol. (14.4 mL, 5.76 mmol, 4.00 equiv.), warmed to 70° C. and stirred at this temperature for 7 h. A second portion of 0.4 M aq. KMnO₄-sol. (7.20 mL, 2.88 mmol, 2.00 equiv.) and tBuOH (5 mL) were added and the mixture was stirred at 70° C. for an additional 17 h. A third portion of 0.4 NI aq. KMnO₄-sol. (7.20 mL, 2.88 mmol, 2.00 equiv.) and tBuOH (5 mL) were added and stirring was continued at 70° C. for an additional 24 h. The reaction mixture was cooled to ambient temperature, treated with ⁱPrOH (5 mL) and stirred overnight. Celite (4 g) was added, stirring was continued for 1 h and the mixture was filtered through Celite. The filtrate was concentrated under reduced pressure to half of its volume, acidified with 3 M aq. HCl-sol. (pH, 2) and extracted with Et₂O (5×10 mL). The combined organic extracts were washed with brine (25 mL), dried over MgSO₄ and evaporated under reduced pressure. The residue was purified by column chromatography (20 g SiO₂, hexane/EtOAc/HOAc 50:50:1→EtOAc/HOAc 100:1) to give 60.2 mg of the mono methyl monocarboxy derivative as a beige powder (173 μmol, 12%), m. p. 145-148° C. and the dicarboxy derivative 13 which was further purified by recrystallisation from EtOAc/hexane to give 345 mg of 13 as a colourless powder (914 μmol, 63%), m. p. 173-175° C. ¹H-NMR (CD₃OD, 400 MHz): δ=0.70-0.95 (m, 6H, CH₂CH₃), 0.90-1.10 (m, 6H, CH₂CH₃), 1.65-1.90 (m, 4H, CH₂CH₃), 1.90-2.15 (m, 4H, CH₂CH₃), 2.13 (s, 3H, CH₃CO), 7.49 (s, 2H, Ph-H). The signals of the CO₂H protons could not be assigned. ¹³C-NMR (CD₃OD, 100 MHz, add. DEPT): δ=7.68, 8.31 (+, CH₂CH₃), 17.73 (+, CH₃CO), 28.48, 29.92 (−, CH₂CH₃), 73.79 (C_{quat}, CCH₂), 123.85 (+, Ph-C), 131.74, 144.84 (C_{quat}, Ph-C), 169.80, 170.63 (C_{quat}, C═O). MS (ESI): negative mode: m/z (%)=376 (100) [M⁻-H]. HRMS (ESI): m/z: calcd. for C₂₀H₂₆NO₆[M⁻-H]: 376.17601, found 376.17490. C₂₀H₂₇NO₆ (377.44): calcd. C, 63.65; H, 7.21; N, 3.71; found: C, 63.38, H, 7.27, N, 3.65.

1,1,3,3-Tetraethyl-5,6-dicarboxylisondoline-2-yloxyl (1): A suspension of the dicarboxylsoindoline 13 (175 mg, 464 μmol, 1.00 equiv.) in H₂O (2 mL) was cooled to 0° C. LiOH (55.6 mg, 2.32 mmol, 5.00 equiv.) was added and the mixture was stirred for 16 h while warming up to ambient temperature. The obtained solution was acidified by addition of 3 M aq. HCl-sol. (pH, 1) and extracted with Et₂O (3×8 mL). The combined organic extracts were treated with PbO₂ (27.7 mg, 116 μmol, 0.25 equiv.) and stirred for 20 min. The mixture was dried over MgSO₄, filtered and evaporated under reduced pressure. The residue was recrystallised from H₂O/MeCN (6:1.5) to give 139 mg of 1 as yellow crystals (416 μmol, 90%). M.p. 186-188° C. MS (EI): m/z (%)=333 (100) [M⁻-H]. HRMS (ESI): m/z: calcd. for C₁₈H₂₃NO₅[M⁻-H]: 333.15762, found 333.157623. C₁₈H₂₄NO₅ (334.39): calcd. C, 64.65; H, 7.23; N, 4.19; found: C, 64.70; H, 7.40; N, 4.27.

### Example 2

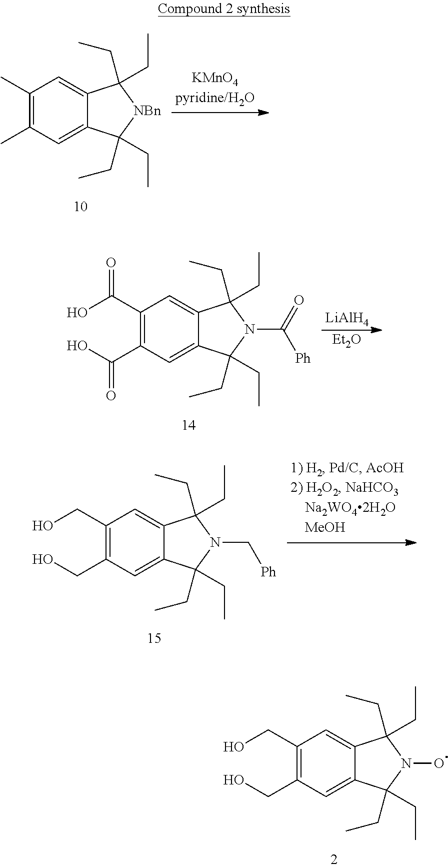

2-Benzoyl-1,1,3,3-tetraethylisoindoline-5,6-dicarboxylic acid (14): A suspension of 2-benzyl-5,6-dimethyl-1,1,3,3-tetraethylisoindoline (10) (1.50 g, 4.29 mmol) and sodium hydroxide (1.00 g, 25.00 mmol) in a mixture of pyridine (30 mL) and water (46 mL) was treated portionwise with solid potassium permanganate (12.00 g, 76.00 mmol). The mixture was heated at reflux for 4 days. Ethanol (30 mL) was added, the mixture filtered and the obtained filtrate concentrated at reduced pressure. The resulting residue was dissolved in water (80 mL), acidified with hydrochloric acid (2 M aqueous solution) and extracted with diethyl ether (5×100 mL). The combined ether layers were dried (anhydrous Na₂SO₄) and concentrated in vacuo to give a white solid (1.35 g, 75%). M.p. 244-246° C. ¹H NMR (400 MHz, CD₃OD): δ=0.7-1.0 (m, 12H, 4×CH₃) 1.6-1.75 (br s, 2 H, CH₂), 1.9-2.1 (br s, 2H, CH₂), 2.4-2.7 (br s, 4H, 2×CH₂), 7.4-7.7 (m, 7H, Ar—H). m/z (%)=422 (100) [M⁻-H]. HRMS (EI): m/z: calcd. for C₂₅H₂₈NO₅[M⁻-H]: 422.1967, found 422.1954.

2-Benzyl-5,6-dihydroxymethyl-1,1,3,3-tetraethylisoindoline (15): 2-Benzoyl-1,1,3,3-tetraethylisoindoline-5,6-dicarboxylic acid (14) (1.0 g, 2.36 mmol) was placed in dry diethyl ether (15 mL) and a solution of lithium aluminium hydride (1.0 M in diethyl ether, 21.24 mL, 21.20 mmol) was added slowly. The mixture was heated at reflux for 3 days, cooled and carefully diluted with water (30 mL). The resulting solution was acidified with hydrochloric acid (2 M aqueous solution) and extracted with chloroform (3×40 mL). The chloroform layers were washed with brine, dried (anhydrous Na₂SO₄) and concentrated in vacuo to give 2-benzyl-5,6-dihydroxymethyl-1,1,3,3-tetraethylisoindoline (15) as an off-white solid (0.71 g, 79%). M.p. 155-158° C. ¹H NMR (400 MHz, CDCl₃): δ=0.77 (t, J=7.4Hz, 12H, 4×CH₃), 1.48-1.58 (m, 4H, 2×CH₂), 1.87-1.95 (m, 4H, 2×CH₂), 4.0 (s, 2H, CH₂), 4.77 (s, 4H, 2×CH₂), 7.04 (s, 2H, Ar—H), 7.1-7.18 (m, 3H, Ar—H), 7.44 (m, 2H, Ar—H). ¹³C NMR (100 MHz, CDCl₃): δ=9.7 (CH₃), 30.3 (CH₂), 30.9 (CH₂), 46.7 (CH₂), 64.8 (CH₂), 71.3 (CH), 124.8 (C), 126.6 (C), 127.8 (C), 129.2 (C), 137.1 (C), 142.1 (C), 145.3 (C). MS (EI): m/z (%)=380 (5) [(M-H)⁺], 352 (75) [(M-C₂H₅)⁺]. HRMS: calcd. for C₂₅H₃₄NO₂ 380.2590; found 380.2586. C₂₅H₃₅NO₂ (381.55): calcd. C, 78.70, H, 9.25, N 3.67; found: C, 78.81; H, 9.25; N, 3.67.

5,6-Dihydroxymethyl-1,1,3,3-tetraethylisoindolin-2-yloxyl (2): An acetic acid (15 mL) solution containing 2-benzyl-5,6-dihydroxymethyl-1,1,3,3-tetraethylisoindoline (15) (0.55 g, 1.44 mmol) and palladium on charcoal (10%, 36 mg, 33.8 μmol, 2.5 mol %) was placed under an atmosphere of hydrogen (50 psi) in a Parr hydrogenator for 7 hours. The solution was filtered through celite and concentrated at reduced pressure. The residue was dissolved in chloroform (30 mL) and washed with sodium hydrogen carbonate (saturated aqueous solution, 2×30 mL) and brine (2×30 mL). The organic layer was dried (anhydrous Na₂SO₄) and concentrated in vacuo. The resulting residue was dissolved in methanol (5 mL). Sodium hydrogen carbonate (0.16 g, 1.9 mmol), sodium tungstate dihydrate (0.07 g, 0.2 mmol) and hydrogen peroxide (30%, 1.4 mL, 12 mmol) were added and the solution was stirred at room temperature for 2 days. Additional sodium tungstate dihydrate (0.1 g, 0.29 mmol) and hydrogen peroxide (30%, 2 mL, 17.1 mmol) were added and the solution was stirred for a further 2 days. Water (20 mL) was added and the mixture was acidified with hydrochloric acid (2 M aqueous solution) and extracted with DCM (3×30 mL). The DCM layers were washed with brine (2×30 mL), dried (anhydrous Na₂SO₄) and concentrated at reduced pressure. Purification by silica gel column chromatography (eluent 70% EtOAc/30% hexane) gave 5,6-dihydroxymethyl-1,1,3,3-tetraethylisoindolin-2-yloxyl (2) as a golden oil which solidified upon standing (0.16 g, 61%). M.p. 114-116° C. MS (EI): m/z (%)=306 (15) [M⁺], 278 (100) [(M-C₂H₅)⁺]. HRMS: calcd. for C₁₈H₂₈NO₃ 306.2069; found 306.2069. C₁₈H₂₈NO₃ (306.42): calcd. C, 70.55; H, 9.21; N, 4.57; found: C, 70.61, H, 9.40, N, 4.44.

### Example 3

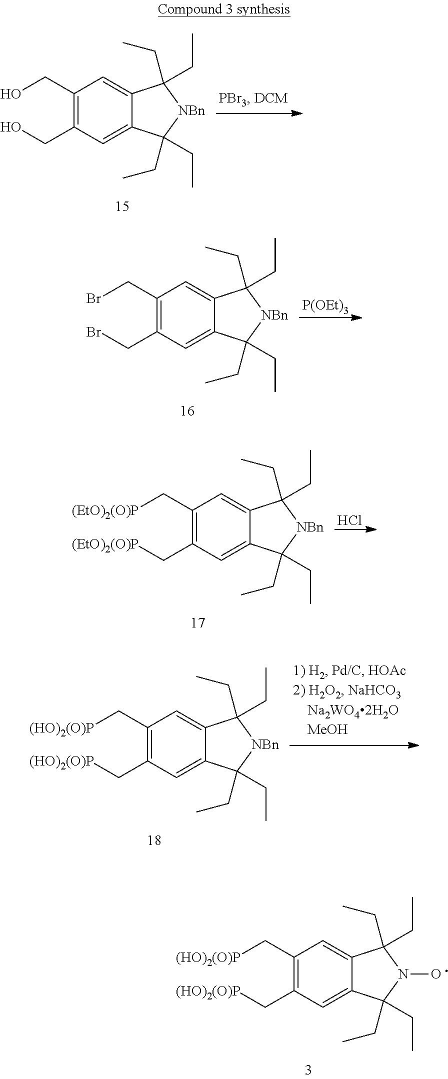

2-Benzyl-5,6-dibromomethyl-1,1,3,3-tetraethylisoindoline (16): Phosphorus tribromide (0.10 mL, 3.10 mmol) was added slowly to an ice-cooled solution of 2-benzyl-5,6-dihydroxymethyl-1,1,3,3-tetraethylisoindoline (15) (0.50 g, 1.31 mmol) in dry DCM (10 mL) under an argon atmosphere. The solution was stirred on ice for 1.5 h, diluted with water (30 mL) and extracted with chloroform (3×30 mL). The organic layers were washed with brine, dried (anhydrous Na₂SO₄) and concentrated at reduced pressure. Purification by silica gel chromatography (eluent 30% DCM/70% hexane) gave 16 as a pale yellow solid (0.32 g, 48%). M.p. 164-166° C. ¹H NMR (400 MHz, CDCl₃): δ=0.72-0.8 (m, 12H, 4×CH₃), 1.48-1.6 (m, 4H, 2×CH₂), 1.85-1.95 (m, 4H, 2×CH₂), 3.99 (s, 2 H, CH₂), 4.71 (s, 4H, 2×CH₂), 7.04 (s, 2H, Ar—H), 7.22-7.34 (m, 3H, Ar—H), 7.41-7.46 (m, 2H, Ar—H). ¹³C NMR (100 MHz, CDCl₃): δ=9.6 (CH₃), 30.2 (CH₂), 30.9 (CH₂), 46.7 (CH₂), 71.4 (C), 125.0 (Ar—C), 126.1 (Ar—C), 126.7 (Ar—C), 127.9 (Ar—C), 129.2 (Ar—C), 134.1 (Ar—C), 146.4 (Ar—C). MS (EI): m/z (%)=478/480/476 (85/43/43) [M⁺-C₂H₅]. HRMS: calcd. for C₂₅H₃₃⁸¹Br₂N, 480.0548; found 480.0537.

Tetraethyl (2-benzyl-1,1,3,3-tetraethylisoindoline-5,6-diyl)bis(methylene)diphosphonate (17): A solution of 2-benzyl-5,6-dibromomethyl-1,1,3,3-tetraethylisoindoline (16) (0.10 g, 0.197 mmol) in triethyl phosphite (85 μL, 0.495 mmol) was heated at 80° C. for 16 h. The excess diethyl phosphite was removed by distillation. Purification of the resulting residue by silica gel chromatography (eluent 100% EtOAc→10% MeOH/90% EtOAc) gave 17 as a golden oil which solified upon standing (0.11 g, 94%). M.p. 81-83° C. ¹H NMR (400 MHz, CDCl₃): δ=0.76 (t, J=7.3Hz, 12H, 4×CH₃), 1.23 (t, J=7.1Hz, 12H, 4×CH₃), 1.45-1.55 (m, 4H, 2×CH₂), 1.85-1.95 (m, 4 H, 2×CH₂), 3.43 (d, J=20.1Hz, 2H, CH₂), 3.92-4.08 (m, 10H, 5×CH₂), 6.95 (d, J=1.9Hz, 2H, Ar—H), 7.2-7.34 (m, 3H, Ar—H), 7.42-7.46 (m, 2H, Ar—H). ³¹P NMR (162 MHz, CDCl₃): δ=27.6. MS (EI): m/z (%)=620 (2) [(M-H)⁺], 592 (100) [(M-C₂H₅)⁺]. HRMS: calcd. for C₃₃H₅₃NO₆P₂ 620.3270; found 620.3264. C₃₃H₅₃NO₆P₂ (621.72): calcd. C, 63.75; H, 8.59; N, 2.25; found: C, 64.03; H, 8.61; N, 2.21.

2-Benzyl-1,1,3,3-tetraethylisoindoline-5,6-diyl)bis(methylene)diphosphonic acid (18): A solution of tetraethyl (2-benzyl-1,1,3,3-tetraethylisoindoline-5,6-diyl)bis(methylene)diphosphonate (17) (0.115 g, 0.185 mmol) was heated to reflux in hydrochloric acid (6 M, 4 mL) for 16 h. The solution was concentrated in vacuo and titrated with ethyl acetate (2×1 mL) to give 18 as a white solid (0.1 g, 92%). M.p. 280-282° C. ¹H NMR (400 MHz, d₆-DMSO): δ=1.68 (t, J=7.2Hz, 12H, 4×CH₃), 2.35-2.45 (m, 4H, 2×CH₂), 2.8-2.9 (m, 4H, 2×CH₂), 4.12 (d, J=20.4Hz, 2H, CH₂), 4.91 (s, 2H, CH₂), 7.90 (s, 2H, Ar—H), 8.13-8.4 (m, 5H, Ar—H). MS (ES): m/z (%)=510 (100) [MH⁺]. HRMS: calcd. for C₂₅H₃₈NO₆P₂ 510.2174; found 510.2176.

5,6-Bis(methylene)diphosphonic acid-1,1,3,3-tetraethylisoindolin-2-yloxyl (3): 2-Benzyl-1,1,3,3-tetraethylisoindoline-5,6-diyl)bis(methylene)diphosphonic acid (18) (85.0 mg, 0.167 mmol) was dissolved in methanol (10 mL) and palladium on carbon (˜20 mg) added. The solution was shaken under an atmosphere of hydrogen gas (50 psi) for 6 h, then filtered through celite and concentrated in vacuo. The resulting residue was dissolved in methanol (5 mL), treated with NaHCO₃ (25 mg, 0.298 mmol), Na₂WO₄.2H₂O (5 mg, 16 μmol) and then hydrogen peroxide (0.1 mL, 30% in H₂O) and the reaction mixture was stirred for 1 d. A second portion of NaHCO₃ (25 mg, 0.298 mmol), Na₂WO₄.2H₂O (5 mg, 16 μmol) and hydrogen peroxide (0.1 mL, 30% in H₂O) was added and stirring was continued for an additional 2 d. The reaction mixture was concentrated to half of its volume, acidified by careful addition of 2 M aq. H₂SO₄ sol. and extracted with diethyl ether (3×10 mL). The combined organic layers were dried over MgSO₄ and evaporated under reduced pressure to give 3 as a white solid (40 mg, 55%); M.P. >250° C. (decomp. MS (EJ): m/z (%)=433 (10) [M-H]⁻. HRMS: Calcd for C₁₈H₂₉NO₇P₂ 433.1419; found: 433.1437.

### Example 4

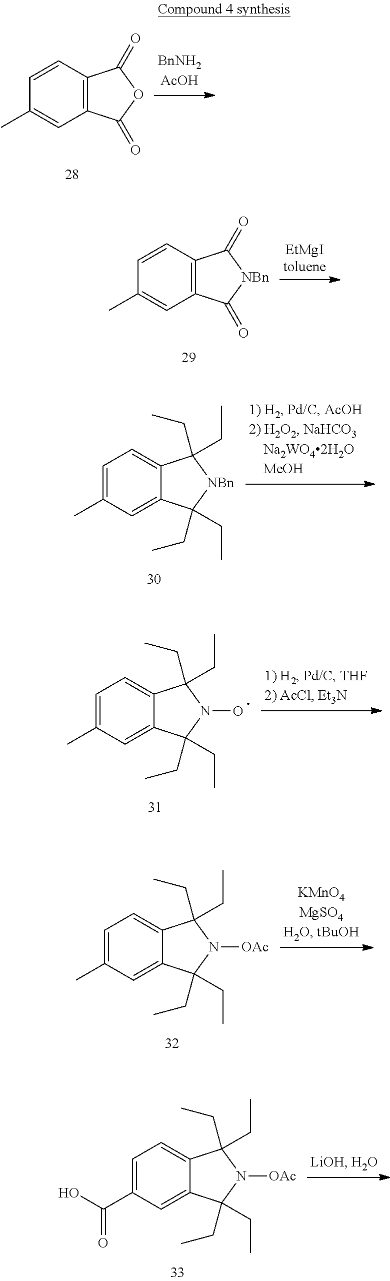

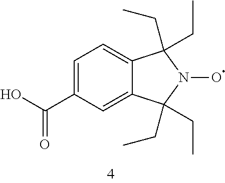

2-Benzyl-5-methylphthalimide (29): Benzyl amine (10.10 mL, 92.60 mmol) was added to a solution of 4-methylphthalic anhydride (28) (10.00 g, 61.70 mmol) in acetic acid (50 mL). The solution was heated to reflux for 1 h and then poured onto ice/water (150 mL) with stirring. The white precipitate was collected by filtration and recrystallised from ethanol to give a fluffy white crystals (14.90 g, 96%). M.p. 128-130° C. ¹H-NMR (CDCl₃, 400 MHz): δ=2.51 (s, 3H, CH₃), 4.85 (s, 2H, CH₂), 7.25-7.36 (m, 3H, Ar—H), 7.42-7.46 (m, 2H, Ar—H), 7.51 (dd, J=7.6, 1.1Hz, 1H, 6-H), 7.66 (s, 1H, 4-H), 7.73 (d, J=7.6Hz, 1H, 7-H). MS (EI): m/z (%)=251 (100) [M⁺]. C₁₆H₁₃NO₂ (251.10): calcd. C, 76.48, H, 5.21, N, 5.57; found: C, 76.20; H, 5.03; N, 5.56.

2-Benzyl-5-methyl-1,1,3,3-tetraethylisoindoline (30): A solution of 2-benzyl-5-methylphthalimide (29) (10.00 g, 40.00 mmol, 1.00 equiv.) in anhydrous toluene (80 mL) was treated with ethyl magnesium iodide [freshly prepared from ethyl iodide (19.20 mL, 24.00 mmol) and magnesium turnings (11.68 g, 48.00 mmol) in Et₂O (100 mL)]. The Et₂O was distilled off via Dean-Stark. The reaction mixture was heated to reflux, stirred for 3 h and then concentrated to about half of its volume. Hexane (4×130 mL) was added, the mixture was filtered through Celite and washed thoroughly with extra hexane (100 mL). The filtrate was passed through a column of basic alumina and concentrated in vacuo to give a colourless oil (4.5 g, 34%). ¹H-NMR (CDCl₃, 400 MHz): S=0.78 (td, J=7.36, 3.04 Hz, 12H, 4×CH₃), 1.45-1.62 (m, 4H, 2×CH₂), 1.85-1.95 (m, 4H, 2×CH₂), 2.37 (s, 3 H, CH₃), 4.00 (s, 2H, CH₂), 6.86 (s, 1H, 4-H), 6.94 (d, J=7.7Hz, 1H, 6-H), 7.02 (d, J=7.7Hz, 1H, 7-H). MS (EI): m/z (%)=336 (50) [MH⁺]. HRMS (ES): calcd. for C₂₄H₃₄N[MH⁺]: 336.2691, found 336.2690.

5-Methyl-1,1,3,3-tetraethylisondoline-2-yloxyl (31): 2-Benzyl-5-methyl-1,1,3,3-tetraethylisoindoline (30) (0.05 g, 1.49 mmol) was dissolved in AcOH (25 mL). Palladium (10% on charcoal, −20 mg) was added and the reaction mixture was shaken under an atmosphere of hydrogen (50 psi in a Parr apparatus) for 3 h. The mixture was filtered through celite and concentrated at reduced pressure. The resulting residue was dissolved in DCM (50 mL) and washed with sodium hydrogen carbonate (saturated aqueous solution, 3×50 mL). The organic phase was dried (anhydrous Na₂SO₄) and concentrated in vacuo to give a yellow oil (0.35 g). The residue was dissolved in MeOH (10 mL), treated with NaHCO₃ (0.13 g, 1.56 mmol) and Na₂WO₄.2H₂O (52.5 mg, 168 μmol), and then hydrogen peroxide solution (30%, 1.15 mL, 11.22 mmol,) and stirred for 1 d. A second portion of NaHCO₃ (0.13 g, 1.56 mmol), Na₂WO₄.2H₂O (52.5 mg, 168 μmol) and hydrogen peroxide solution (30%, 1.15 mL, 11.22 mmol,) was added and stirring was continued for an additional 2 d. Water (20 mL) was added and the mixture extracted with DCM (3×20 mL). The combined organic layers were washed with sulphuric acid (2 M aqueous solution, 2×25 mL), dried (anhydrous Na₂SO₄) and evaporated under reduced pressure. The residue was purified by silica gel chromatography (eluent 100% DCM) to give 31 as an orange oil (0.24 g, 61%). MS (ES): m/z (%)=283 (20) [MNa⁺], 261 (2) [MH⁺]. HRMS (ES): m/z: calcd. for C₁₇H₂₇NO [MH⁺]: 261.2093, found 261.2091.

N-Acetyloxy-5-methyl-1,1,3,3-tetraethylisoindoline (32): A solution of 5-methyl-1,1,3,3-tetraethylisondoline-2-yloxyl (31) (1.00 g, 3.84 mmol) in dry THF (20 mL) was treated with palladium (102 mg, 96.0 μmol, 10% on charcoal) and stirred under a balloon of H₂ for 30 min. The reaction mixture was cooled to 0° C., Et₃N (1.07 mL, 7.68 mmol) and acetyl chloride (0.68 mL, 9.60 mmol) were added and the mixture was stirred at 0° C. for 30 min. The cooling bath was removed and stirring was continued for an additional 1 h. Ar was bubbled over the mixture for 10 min. The reaction mixture was filtered through celite and concentrated in vacuo. Water (50 mL) was added and the mixture was extracted with EtOAc (3×50 mL). The combined organic extracts were dried over Na₂SO₄ and evaporated at reduced pressure. The resulting residue was purified by silica gel chromatography (eluent DCM/hexane 1:1, sample loaded in DCM) to give 32 as a colourless oil which solidified upon standing (1.10 g, 95%). M.p. 76-78° C. ¹H-NMR (CDCl₃, 400 MHz): δ=0.75-0.85 (m, 6H, 2×CH₃), 0.9-1.0 (m, 6H, 2×CH₃), 1.6-2.05 (m, 8H, 4×CH₂), 2.52 (s, 3H, CH₃), 2.75 (s, 3H, CH₃), 6.87 (s, 1H, 4-H), 6.96 (d, J=7.7Hz, 6-H), 7.07 (d, J=7.7Hz, 7-H). ¹³C-NMR (CDCl₃, 100 MHz): δ=8.6 (CH₃), 9.4 (CH₃), 19.4 (CH₃), 21.6 (CH₃), 28.9 (CH₂), 30.3 (CH₂), 73.5 (C), 73.6 (C), 123.4 (CH), 124.0 (CH), 127.5 (CH), 136.2 (C), 138.7 (C), 141.7 (C), 170.6 (C═O). MS (ES): m/z (%)=326 (40) [MNa⁺], 304 (5) HRMS (ES): m/z: calcd. for C₁₉H₃₀NO₂ [MH⁺]: 304.2277, found 304.2280. C₁₉H₂₉NO₂ (303.22): calcd. C, 75.21; H, 9.63; N, 4.62; found: C, 75.10, H, 9.69, N, 4.53.

N-Acetyloxy-5-carboxy-1,1,3,3-tetraethylisoindoline (33): N-Acetyloxy-5-methyl-1,1,3,3-tetraethylisoindoline (32) was dissolved in tert-butanol (17 mL) and warmed to 40° C. Magnesium sulphate (0.30 g, 1.24 mmol) and potassium permanganate solution (0.4 M in water, 25 mL, 10.00 mmol) were added and the mixture was heated at 70° C. for 24 h. The solution was cooled, treated with isopropanol (10 mL) and stirred for 16 h. The mixture was filtered through celite. The filtrate was concentrated by half, acidified with hydrochloric acid (2 M aqueous solution) and extracted with diethyl ether (4×15 mL). The organic layers were dried (anhydrous Na₂SO₄) and concentrated at reduced pressure. Purification of the resulting residue by silica gel chromatography (eluent DCM/EtOAc 3:2) gave 33 as a white solid. Recrystallisation from hexane/EtOAc gave white prisms (0.51 g, 62%). M.p. 168-170° C. ¹H-NMR (CDCl₃, 400 MHz): δ=0.81 (br. s, 6H, 2×CH₃), 0.98 (br s., 6H, 2×CH₃), 1.62-1.86 (m, 4H, 2×CH₂), 1.88-2.1 (m, 4H, 2×CH₂), 2.13 (s, 3 H, CH₃), 7.18 (d, J=8.0Hz, 1H, 7-H), 7.82 (1H, 4-H), 8.04 (d, J=8.0Hz, 1H, 6-H). ¹³C-NMR (CDCl₃, 100 MHz): δ=8.5 (CH₃), 9.3 (CH₃), 19.3 (CH₃), 28.9 (CH₂), 30.2 (CH₂), 73.7 (CH), 74.0 (CH), 123.7 (CH), 125.4 (CH), 128.0 (C), 128.9 (CH), 142.3 (C), 148.2 (C), 170.3 (C═O), 172.1 (C═O). MS (ES): m/z (%)=332 (100) [(M-H)⁻]. HRMS (ES): m/z: calcd. for C₁₉H₂₆NO₄ [(M-H)⁻]: 332.1862, found 332.1870. C₁₉H₂₇NO₄ (333.42): calcd. C, 68.44; H, 8.16; N, 4.20; found: C, 68.48; H, 8.24; N, 4.13.

5-Carboxy-1,1,3,3-tetraethylisoindolin-2-yloxyl (4): N-Acetyloxy-5-carboxy-1,1,3,3-tetraethylisoindoline (33) (0.20 g, 0.60 mmol) was suspended in water (4 mL) and the mixture cooled on ice. Lithium hydroxide (71 mg, 2.99 mmol) was added, the ice-bath was removed and the mixture was stirred at room temperature for 16 h. The resulting yellow solution was acidified with hydrochloric acid (2 M aqueous solution) and extracted with diethyl ether (3×15 mL). The combined ether layers were treated with lead oxide (71 mg, 0.30 mmol) and stirred for 20 min. The solution was dried (Na₂SO₄), filtered and concentrated in vacuo to give a yellow oil which solidified upon standing. Recrystallisation from acetonitrile gave yellow crystals (0.15 g, 85%). M.p. 97-99° C. MS (ES): m/z (%)=289 (100) [(M⁻H)⁻]. HRMS (ES): m/z: calcd. for C₁₇H₂₃NO₃ [(M-H)⁻]:289.1678, found 289.1679. C₁₇H₂₄NO₃ (290.18): calcd. C, 70.32; H, 8.33; N, 4.82; found: C, 70.29, H, 8.35, N 4.77.

### Example 5

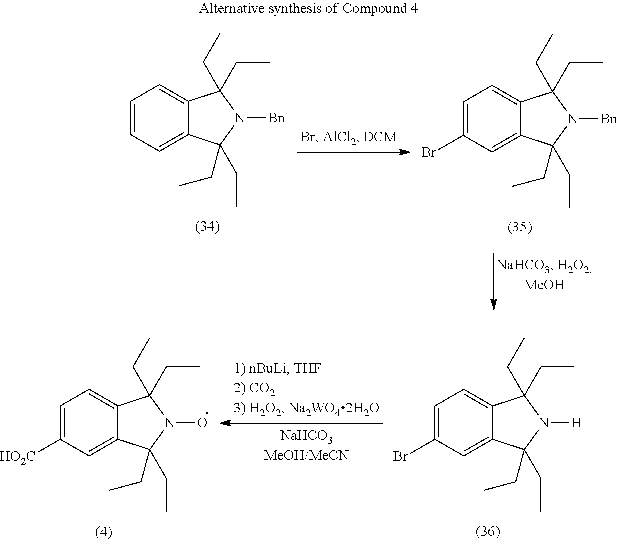

2,5-Dibromo-1,1,3,3-tetraethylisoindoline (35): 2-Benzyl-1,1,3,3-tetraethylisoindoline (34) (5.00 g, 15.60 mmol) was dissolved in DCM (50 mL) under argon. The solution was cooled on ice and a solution of bromine (1.80 mL, 35 mmol) in DCM (38 mL) was added dropwise, followed by the immediate addition of aluminium trichloride (7.50 g, 56.30 mmol). The solution was stirred at 0° C. for 1 h and then poured onto ice (50 mL). After 30 min of vigorous stirring, the mixture was basified with sodium hydroxide (5 M aqueous solution) and extracted with DCM (3×60 mL). The DCM layers were washed with brine (2×60 mL) and concentrated at reduced pressure to give an orange oil. Purification by column chromatography (eluent DCM/hexane, 3:7) gave 2,5-dibromo-1,1,3,3-tetraethylisoindoline (35) as a pale orange oil (1.90 g, 31%) containing trace amounts of (<5% by ¹H NMR) 2,5,6-tribromo-1,1,3,3-tetraethylisoindoline. ¹H NMR (400 MHz, CDCl₃): δ=0.85-0.92 (m, 12H, 4×CH₃) 1.6-1.79 (m, 8H, 4′×CH₂), 6.94 (d, J=8.1Hz, 1H, H7), 7.2 (d, J=1.8Hz, 1H, H6), 7.33 (dd, J=8.1Hz, 1.8Hz, 1H, H4) ppm. ¹³C NMR (100 MHz, CDCl₃): δ=8.8 (CH₃), 33.58 (CH₂), 33.62 (CH₂), 68.2 (C_{quat}), 68.3 (C_{quat}) 120.3 (C_{quat}), 124.0 (CH), 125.6 (CH), 129.6 (CH), 145.5 (C_{quat}), 150.0 (C_{quat}) ppm. MS (EI): m/z (%)=389 (40), 387/391 (20) [M⁺].

5-Bromo-1,1,3,3-tetraethylisoindoline (36): Sodium hydrogen carbonate (0.40 g, 4.77 mmol) was added to a solution of 2,5-dibromo-1,1,3,3-tetraethylisoindoline (35) (1.00 g, 2.57 mmol) in methanol (10 mL). Hydrogen peroxide (30% aqueous solution, ≈15 mL) was then added slowly until the observed effervescence ceased (ensuring that some sodium hydrogen carbonate remained). The solution was acidified with hydrochloric acid (2M aqueous solution) and extracted with DCM (3×50 mL). The DCM layers were dried (anhydrous MgSO₄) and concentrated in vacuo to give 5-bromo-1,1,3,3-tetraethylisoindoline (36) as a pale yellow solid (0.78 g, 98%) containing trace amounts (<5% by ¹H NMR) of 5,6-dibromo-1,1,3,3-tetraethylisoindoline. M.p. >250° C. (decomp). ¹H NMR (400 MHz, CDCl₃): δ=1.45 (br. s, 12H, 4×CH₃), 2.1-2.25 (m, 4H, 2×CH₂), 2.3-2.45 (m, 4H, 2×CH₂), 7.02 (d, J=8.06Hz, 1H, H7), 7.26 (d, J=1.63Hz, 1H, H6), 7.49 (dd, J=8.04, 1.63Hz, 1H, H4) ppm. ¹³C NMR (100 MHz, CDCl₃): δ=8.76 (CH₃), 8.8 (CH₃), 30.73 (CH₂), 30.78 (CH₂), 75.9 (C_{quat}), 76.0 (C_{quat}) 122.4 (C_{quat})_{,} 124.7 (CH), 126.3 (CH), 131.7 (CH), 139.8 (C_{quat}), 143.1 (C_{quat}) ppm. MS (ES): m/z (%)=310/312 (100) [MH⁺]. HRMS: calcd for C₁₆H₂₅⁸¹Br₂N [MH⁺] 312.1144; found 312.1150. HRMS calcd for C₁₆H₂₅⁷⁹Br₂N [MH⁺] 310.1170; found 310.1163.

5-Carboxy-1,1,3,3-tetraisoindol-2-yloxyl (4): n-Butyllithium (1.6 M in hexanes, 5.76 mL, 9.22 mmol) was added slowly to a solution of 5-bromo-1,1,3,3-tetraethylisoindoline (36) (1.30 g, 4.19 mmol) in dry THF (12 mL) at −78° C. under argon. After stirring for 10 min. the solution was poured onto a slurry of powdered dry ice and dry THF (40 mL total). The mixture was stirred until it reached room temperature and then concentrated to dryness. The resulting residue was dissolved in diethyl ether (50 mL) and extracted with hydrochloric acid (2M aqueous solution, 2×30 mL). The ether layers were dried (anhydrous MgSO₄) and concentrated in vacuo. The resulting residue was dissolved in a mixture of methanol (20 mL), water (10 mL) and acetonitrile (15 mL). The solution was treated with sodium hydrogen carbonate (0.29 g, 3.40 mmol) and sodium tungstate dehydrate (0.13 g, 0.38 mmol), followed by the addition of hydrogen peroxide solution (30%, 2.5 mL). The solution was stirred at ambient temperature for 24 h, extra hydrogen peroxide solution (30%, 0.5 mL) was added and the solution stirred for a further 72 h. The mixture was basified with sodium hydroxide (2 M aqueous solution) and extracted with diethyl ether (3×60 mL) and the ether layers discarded. The basic layers were acidified with hydrochloric acid (2 M, aqueous solution) and extracted with diethyl ether (3×60 mL). The ether layers were dried (anhydrous MgSO₄) and concentrated at reduced pressure to give a yellow oil which solidified upon standing (0.25 g, 21%). Recrystallisation from acetonitrile gave yellow crystals; M.p. 97-99° C. MS (ES): m/z (%)=289 (100) [M-H]⁻. HRMS (ES): m/z: calcd for C₁₇H₂₃NO₃ [(M-H)]⁻: 289.1678; found 289.1679. C₁₇H₂₄NO₃ (290.13): calcd C, 70.32; H, 8.33; N, 4.82; found C, 70.29, H, 8.35, N, 4.77.

### Example 6

### Biological Assessment Protocols

A-T Cell Survival Assay Procedure

### BACKGROUND

A-T cells are known to be radiosensitive, and current research has shown the link between the radiosensitive nature of A-T cells and heightened oxidative stress levels due to the lack of, or inactivation of, ataxia telangiectasia mutated (ATM) proteins. Radiation causes damage mainly by the formation of ROS in biological systems, and the lack of mechanisms in the A-T cells to withstand damage leads to cell death or poor cell development.

Lymphoblastoid Cell Lines (LCL) of A-T cells and standard cells were used, which were incubated and cultured in the standard conditions. The cell lines of both A-T cells and standard cells were initially divided into four batches, the first batch was not irradiated and not exposed to nitroxide, second batch was irradiated in the absence of nitroxide, the third batch was not irradiated but was exposed to the nitroxide, and the last batch was irradiated in the presence of the nitroxide. All four aliquots were taken from the cultured medium and counted to ensure consistent numbers of cells were present in each aliquot used in the assay. In some experiments comparative experiments were performed with the known nitroxide antioxidant CTMIO. Cells that were treated by the nitroxide studied were exposed for at least six hours prior to the irradiation at the lethal dose of 4 Gy. After dilution, the final concentrations of the nitroxides were made to be 100 μM. Previous experiments have shown that nitroxides generally have minimal toxicity at the concentration of 100 μM, but to confirm this, cell growth was monitored in the presence of the nitroxides but without exposure to irradiation. From this it was evident that there was no significant cytotoxicity with any of the compounds studied.

The concentrations of the cell cultures were monitored every 24 hours after irradiation, for four days. This was conducted via a Trypan blue based assay using a bright-line hemacytometer and an optical microscope. Trypan blue solution enables the discrimination of live cells from dead cells, as the dye is completely absorbed by dead cells, but not by live cells. Under a bright-line hemacytometer, a glass slide specially designed to determine cell concentrations, the blue dead cells fade into the background, enabling the selective count of live cells under an optical microscope. All counts were conducted in triplicates, to ensure statistical validity of results. The cell suspensions were thoroughly mixed before any transfer, to ensure equal concentrations.

Cell survivability was determined by the ratio of cells irradiated to the cells without irradiation. As the cells have the same growth rates under the same conditions, the ratio enables the survivability to be estimated. Errors were calculated with 95% confidence intervals.

The control cell line JHP and A-T cell line KT were obtained from a healthy patient and an A-T patient, respectively. The Epstein-Barr virus converted blood cells of the patients to Lymphoblastoid Cell Lines (LCL). Cells were cultured in RPIM 1640 growth media, containing 10% foetal calf serum (FCS), incubated at 37° C. at 5% CO₂/air atmosphere. Trypan Blue solution used consisted of 0.4% w/w Trypan Blue in Hanks solution. The dye solution was allowed to settle at least 24 hours before use. Cells were observed under an optical microscope with 10× optical zoom. A bright-line Hemacytometer was used to facilitate cell counts.

The nitroxides investigated were first dissolved in 0.1 mL DMSO to prepare 0.1M stocks. The 0.1M stock solution was diluted with the growth media make 1 mL of 10 mM nitroxide stock.

A cell count of the cell culture was conducted prior to all experiments, in order to measure the concentration of cells. Each experiment required dilution to obtain approx. 2×10⁵ cells/mL. Appropriate amounts of the cell culture were transferred to culture tubes in order to attain approximately 2×10⁵ cells/cm³ after dilution to 5 mL.

The cell stock was transferred to culture tubes, in which the amount was that which enabled 5 mL of approx. 2×10⁵ cells/mL. Nitroxide stock solution (50 μL of 10 mM) was added to the cell suspension such that the final concentration of the nitroxide in the tube after dilution to 5 mL was 100 μM.

Before dilution, the cell suspensions were irradiated at radiation doses, varying from 0 Gy to 4 Gy. After irradiation, the cell cultures were diluted to 5 cm⁵, and incubated. Prior to cell counts, to a vial, 0.1 mL of Trypan Blue solution and 0.5 mL of the well-mixed cell suspension were added. The mixture was allowed to sit for 2-3 minutes, to enable the dyes to equilibrate. The cells were counted every 24 hours after irradiation for 4 days.

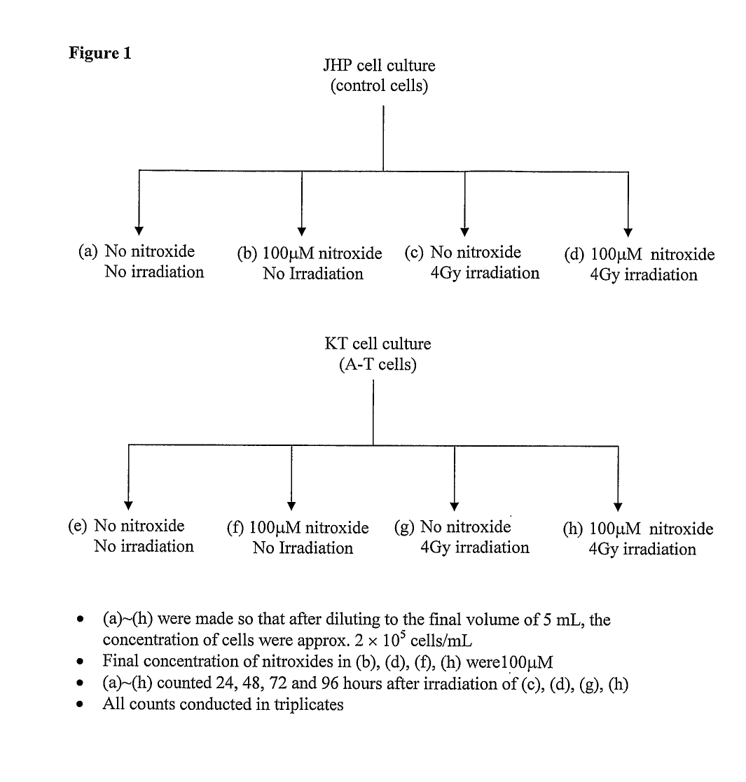

A summary of the experimental protocol of the cell survival assay is shown in FIG. 1. the survivability of cells can be calculated from the following equations:

Survivability of control cells in the absence of antioxidants=[(c)÷(a)/100]%

Survivability of control cells in the absence of antioxidants=[(d)÷(b)/100]%

Survivability of A-T cells in the absence of antioxidants=[(g)÷(e)/100]%

Survivability of A-T cells in the presence of antioxidants=[(h)÷(f)/100]%

Assays were also performed to assess the toxicity of the antioxidant isoindoline nitroxide compounds where the KT cells were exposed to the nitroxide compound in the absence of irradiation and the growth of the cells monitored over 4 days.

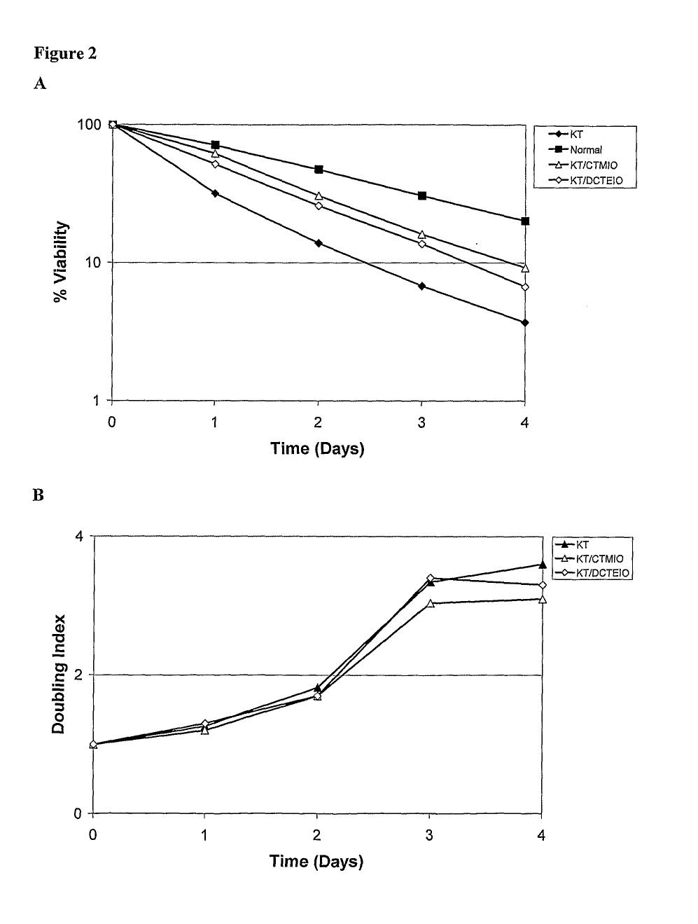

The survivability or viability of cells after exposure to irradiation in the presence or absence of an isoindoline nitroxide antioxidant are shown in FIGS. 2 to 6.

FIG. 2A shows that Compound (1) (DCTEIO) improves survival of KT cells in the presence of irradiation in a similar manner to CTMIO. FIG. 2B shows the growth of KT cells in the presence of DCTEIO or CTMIO.

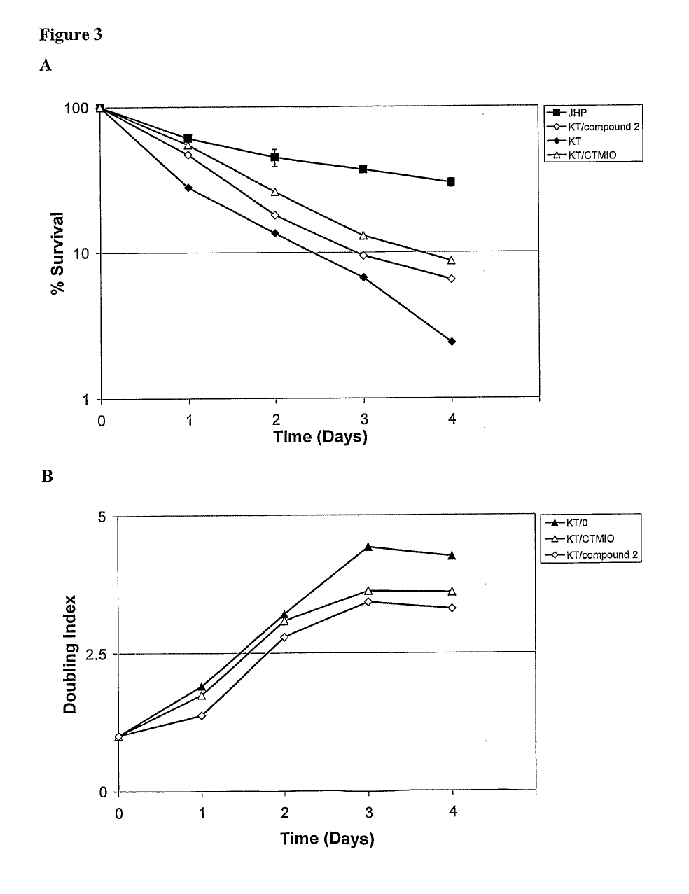

FIG. 3A shows that Compound (2) improves survival of KT cells in the presence of irradiation in a similar manner to CTMIO. FIG. 3B shows the growth of KT cells in the presence of compound (2) or CTMIO.

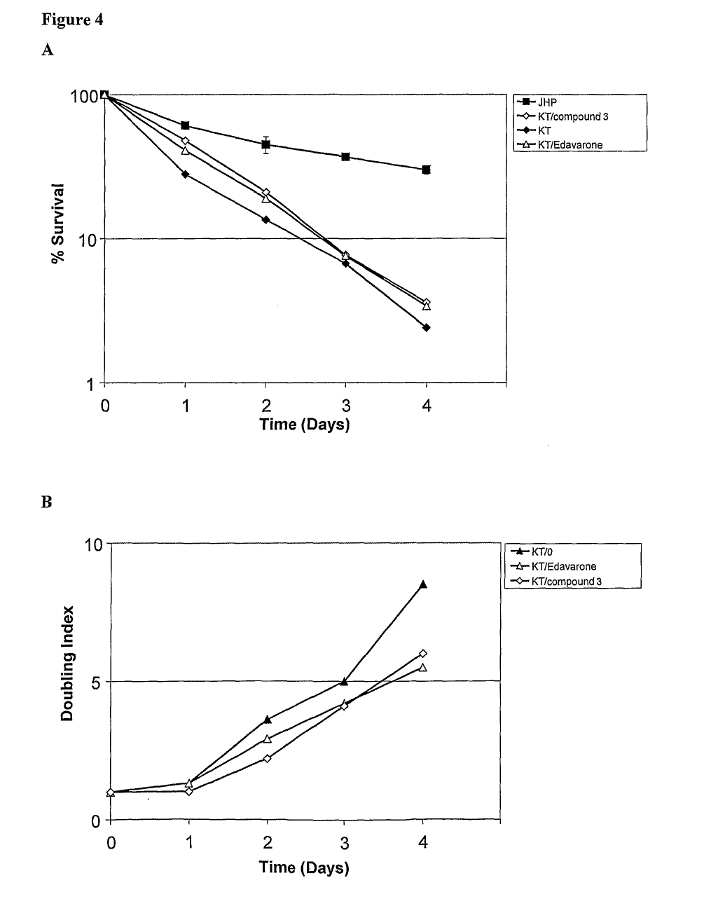

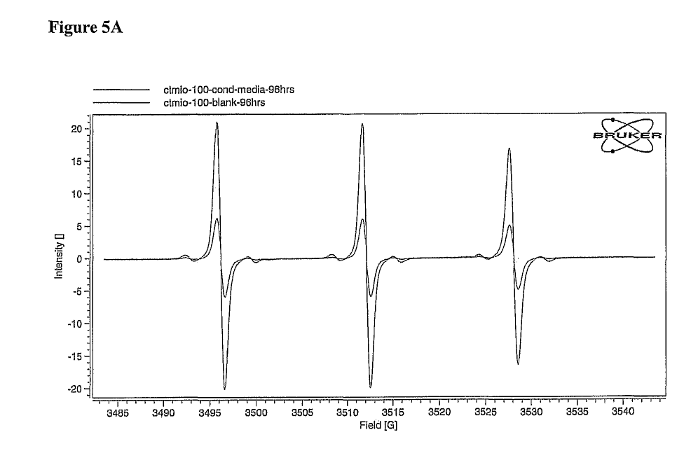

FIG. 4A shows that Compound (3) improves survival of KT cells in the presence of irradiation. FIG. 4B shows the growth of KT cells in the presence of Compound (3).

### Example 7

### Biostability of Compound (1)

PC-3 cells were seeded in tissue culture flasks (surface area ˜80 cm2). The cells were allowed to attach overnight. Following this, the cells were then treated with the desired concentration of CTMIO or DCTEIO (Compound 1) or vehicle (DMSO alone) i.e. 12 mL of media containing different nitroxide or vehicle was added to each flask. 5 mL of conditioned media (i.e. treatment media that had been incubated with the cells) were removed at 24 h and 96 h. At the 96 h time point, cells in the tissue culture flask were ˜100% confluent (i.e. the surface of the flask was covered by a monolayer of cells). The conditioned media that was removed from the flasks was placed in a falcon and centrifuged at 4° C. to remove any cellular debris that might have been present. The supernatant was then transferred to a new tube and the lid parafilmed to make it as airtight as possible. The tubes of conditioned media were stored at −20° C. until Electron Paramagnetic Resonance (EPR) analysis. Hence, all tubes, at the different concentrations and time points were analysed at the same time. The samples were run at X-band on a Bruker ELEXSYS E 580 FT/CW X-Band spectrometer at room temperature.

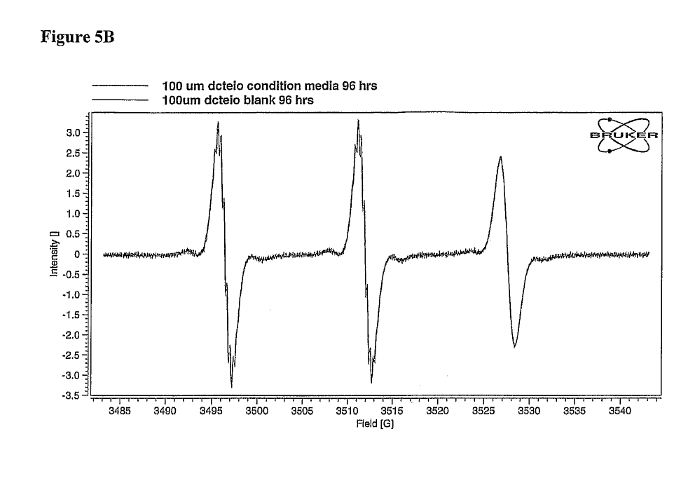

The EPR results are summarised in FIGS. 5A and 5B. FIG. 5A shows the metabolic conversion of CTMIO to the non-radical metabolite hydroxylamine. The larger three peak feature represents the signal strength of the nitroxide run in cellular condition media which, in the absence of cells, shows no change or reduction in strength. This larger three peak signal is superimposed on the smaller three peak feature which represents the signal strength arising from the CTMIO after 96 hours of exposure to the cells, demonstrating a significant reduction in signal strength arising from metabolic conversion to the non-radical hydroxylamine.

Notably this signal loss is not replicated with DCTEIO. It can be seen in FIG. 5B that the same signal strength for DCTEIO is present without cells and at t=96 hr cell exposure, demonstrating that the metabolic reduction of the DCTEIO is very slow and the free radical character remains unchanged under this timeframe.

### Example 8

### Solubility and Permeability of DCTEIO

The solubility and permeability of CTMIO and DCTEIO (Compound 1) were assessed using the shake flask method and UV/vis spectroscopy to measure distribution of the solute as follows:

A sample of octanol containing DCTEIO (3.34 mg, 1 mM) was added to a separating funnel with 10 mL water. The separating funnel was shaken for 5 minutes and the mixture was allowed to separate for 16 hours. Each phase (0.5 mL) was diluted with 5 mL of like solvent, water phase diluted with water and n-octanol phase diluted with n-octanol. This protocol was repeated for CTMIO. The absorbance of each sample was measured by UV/vis spectroscopy at 230 nm. The Log P value was calculated by the following equation:

$$Log P = log \frac{Abs Octanol at 230 nm}{Abs Water at 230 nm}$$

### Results:

DCTEIO Log P=0.57

CTMIO Log P=−0.47

DCTEIO had a solubility within the acceptable range (Log P −0.4-5.6) for small drug like molecules.

1. A method of reducing oxidative stress in a cell comprising exposing the cell to an effective amount of a compound of formula (II): 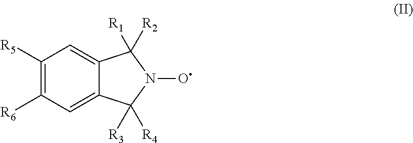
wherein
each of R₁, R₂, R₃ and R₄ are independently selected from —C₁-C₆alkyl, —C₂-C₆alkenyl, —C₂-C₆alkynyl, —C₃-C₈cycloalkyl, aryl, heterocyclyl and heteroaryl wherein at least one of R₁, R₂, R₃ and R₄ is not methyl;
R₅ is selected from hydrogen, —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₆ is selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted;
or a pharmaceutically acceptable salt thereof.

2. A method according to claim 1 wherein R₅ is selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇.

3. A method according to claim 1 or claim 2 wherein R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H and —C₀-C₆alkylCONH₂.

4. A method according to claim 1 wherein at least one of R₅ and R₆ is selected from —CO₂H, —NH₂, —OH, —CH₂OH, —CH₂PO₃H₂ and heterocyclyl.

5. A method according to claim 4 wherein one of R₅ and R₆ is a pyrazolyl or tetrazolyl group.

6. A method according to claim 1 wherein one or more of R₁ to R₄ are C₁-C₆alkyl.

7. A method according to claim 6 wherein one or more of R₁ to R₄ are selected from C₂ and C₃ alkyl.

8. A method according to claim 7 wherein one or more of R₁ to R₄ are selected from ethyl, propyl and isopropyl.

9. A method according to claim 8 wherein one or more of R₁ to R₄ are selected from ethyl.

10. A method according to claim 1 wherein all of R₁ to R₄ are selected from C₁-C₆ alkyl.

11. A method according to claim 10 wherein all of R₁ to R₄ are selected from C₂ or C₃ alkyl.

12. A method according to claim 11 wherein all of R₁ to R₄ are selected from ethyl, propyl and isopropyl.

13. A method according to claim 12 wherein all of R₁ to R₄ are ethyl.

14. A method according to claim 1 wherein the compound of formula (II) is a compound in which all of R₁ to R₄ are ethyl and R₅ and R₆ are both —CO₂H, —CH₂OH or —CH₂PO₃H₂ or where R₅ is hydrogen and R₆ is —CO₂H.

15. A method according to claim 1 wherein the compound of formula (II) is 1,1,3,3-tetraethyl-5,6-dicarboxylisoindoline-2-yloxyl.

16. A method of treating or preventing a disease or disorder related to oxidative stress comprising administering to a subject an effective amount of a compound of formula (II) as defined in claim 1.

17. A method of protecting a subject from oxidative stress upon exposure to ionising radiation comprising administering to the subject an effective amount of a compound of formula (II) as defined in claim 1.

18. A method according to claim 17 wherein the exposure to ionising radiation occurs during therapy with ionising radiation or radiotherapy.

19. (canceled)

20. A compound of formula (I): 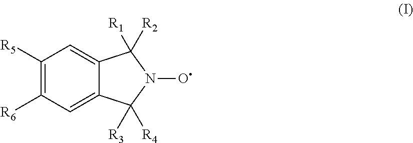
wherein
each of R₁, R₂, R₃ and R₄ are independently selected from —C₁-C₆alkyl, —C₂-C₆alkenyl, —C₂-C₆alkynyl, —C₃-C₈cycloalkyl, aryl, heterocyclyl and heteroaryl wherein at least one of R₁, R₂, R₃ and R₄ is not methyl;
R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H, —C₀-C₆alkylCONH₂, —C₀-C₆alkylCONHSO₂R₇, —C₀-C₆alkylSO₂NHCOR₇, —C₀-C₆alkylSO₂NHCONHR₇, —C₀-C₆alkylSO₂NHR₇, —C₀-C₆alkylNHSO₂R₇, —C₂-C₆alkenylCO₂H, —C₂-C₆alkenylNH₂, —C₂-C₆alkenylOH, —C₂-C₆alkenylPO₃H₂, —C₂-C₆alkenylhalo, —C₂-C₆alkenylNO₂, —C₂-C₆alkenylCN, —C₂-C₆alkenylheterocyclyl, —C₂-C₆alkenylheteroaryl, —C₂-C₆alkenylSO₃H, —C₂-C₆alkenylCONH₂, —C₂-C₆alkenylCONHSO₂R₇, —C₂-C₆alkenylSO₂NHCOR₇, —C₂-C₆alkenylSO₂NHCONHR₇, —C₂-C₆alkenylSO₂NHR₇ and —C₀-C₆alkylNHSO₂R₇;
R₇ is selected from hydrogen, —C₁-C₆alkyl, —C₂-C₆alkenyl, —C₂-C₆alkynyl, —C₃-C₈cycloalkyl, aryl, heterocyclyl and heteroaryl;
wherein each alkyl, alkenyl, alkynyl, cycloalkyl, aryl, heterocyclyl and heteroaryl group may be optionally substituted;
or a pharmaceutically acceptable salt thereof.

21. A compound according to claim 20 wherein R₅ and R₆ are independently selected from —C₀-C₆alkylCO₂H, —C₀-C₆alkylNH₂, —C₀-C₆alkylOH, —C₀-C₆alkylPO₃H₂, —C₀-C₆alkylhalo, —C₀-C₆alkylNO₂, —C₀-C₆alkylCN, —C₀-C₆alkylheterocyclyl, —C₀-C₆alkylheteroaryl, —C₀-C₆alkylSO₃H and —C₀-C₆alkylCONH₂.

22. A compound according to claim 20 wherein at least one of R₅ and R₆ is selected from —CO₂H, —NH₂, —OH, —CH₂OH, —CH₂PO₃H₂ and heterocyclyl.

23. A compound according to claim 20 wherein one of R₅ and R₆ is a pyrazolyl or tetrazolyl group.

24. A compound according to claim 20 wherein one or more of R₁ to R₄ are C₁-C₆alkyl.

25. A compound according to claim 24 wherein one or more of R₁ to R₄ are selected from C₂ and C₃ alkyl.

26. A compound according to claim 25 wherein one or more of R₁ to R₄ are selected from ethyl, propyl and isopropyl.

27. A compound according to claim 26 wherein one or more of R₁ to R₄ are selected from ethyl.

28. A compound according to claim 20 wherein all of R₁ to R₄ are selected from C₂-C₆ alkyl.

29. A compound according to claim 28 wherein all of R₁ to R₄ are selected from C₂ or C₃ alkyl.

30. A compound according to claim 29 wherein all of R₁ to R₄ are selected from ethyl, propyl and isopropyl.

31. A compound according to claim 30 wherein all of R₁ to R₄ are ethyl.

32. A compound according to claim 20 wherein the compound of formula (I) is a compound in which all of R₁ to R₄ are ethyl and R₅ and R₆ are both —CO₂H, —CH₂OH or —CH₂PO₃H₂.

33. A compound of formula (I) according to claim 20 which is 1,1,3,3-tetraethyl-5,6-dicarboxylisoindoline-2-yloxyl.

34. A pharmaceutical composition comprising a compound of formula (II) according to claim 1 together with a pharmaceutically acceptable carrier or excipient.

35. A pharmaceutical composition comprising a compound of formula (I) according to claim 20 together with a pharmaceutically acceptable carrier or excipient.
